(19) United States
(12) Patent Application Publication
(10) Pub. No.: US 2024/0360107 A1
(43) Pub. Date: Oct. 31, 2024

(54) AN EIF4A INHIBITOR WITH A NOVEL MECHANISM OF ACTION

(71) Applicant: ARIZONA BOARD OF REGENTS ON BEHALF OF THE UNIVERSITY OF ARIZONA, Tucson, AZ (US)

(72) Inventors: Eli Chapman, Tucson, AZ (US); Christopher Zerio, Tucson, AZ (US)

(21) Appl. No.: 18/687,664

(22) PCT Filed: Aug. 30, 2022
(86) PCT No.: PCT/US22/75686

§ 371 Date: Feb. 28, 2024

Related U.S. Application Data
(60) Provisional application No. 63/238,456, filed on Aug. 30, 2021.

(51) Int. Cl. C07D 405/04; A61K 31/4709

(52) U.S. Cl. CPC C07D 405/04; A61K 31/4709

(57) ABSTRACT

The present invention features inhibitors of the eIF4A enzyme. These inhibitors have a novel mechanism for inhibiting the eIF4A enzyme by occupying a binding pocket within the eIF4A RNA-binding groove, thereby perturbing RNA binding, blocking ATP hydrolysis, and, consequently, inhibiting RNA helicase activity. Thus, the compounds of the present invention are RNA-competitive, ATP-uncompetitive elF4A inhibitors that directly bind elF4A and inhibit the protein.

### CROSS-REFERENCES TO RELATED APPLICATIONS

This application claims benefit of U.S. Provisional Application No. 63/238,456 filed Aug. 30, 2021, the specification of which is incorporated herein in its entirety by reference.

### STATEMENT REGARDING FEDERALLY SPONSORED RESEARCH OR DEVELOPMENT

This invention was made with government support under Grant No. T32 GM008804 awarded by National Institutes of Health. The government has certain rights in the invention.

### FIELD OF THE INVENTION

The present invention relates to inhibitors of the eIF4A enzyme. In some embodiments, the compounds of the present invention may be useful for the treatment of lymphoma.

### BACKGROUND OF THE INVENTION

Protein synthesis through messenger RNA (mRNA) translation is a strictly controlled process. Dysregulation of this operation is a core feature of cancer, as many pathways that are crucial for oncogenesis are mediated by translational control. The most tightly regulated step of protein biosynthesis is the initiation of cap-dependent translation, in which initiation factors bind to the 5-prime (5′) 7-methylguanosine (m⁷G) cap of mature mRNA to launch translation of open reading frames. Many oncogenes, including those involved in tumor cell proliferation, growth, and angiogenesis depend directly on m⁷G-cap factors for their translation. Conversely, translation of many non-oncogenic genes is cap-independent. Targeting specific cap-dependent translation factors that are altered in expression or activity in human cancers and not involved in translation of non-oncogenic housekeeping genes offers great promise for the development of a new generation of cancer therapeutics.

Cap-dependent translation is driven by the heterotrimeric eukaryotic initiation factor (eIF)4F complex, which catalyzes ribosome recruitment to mRNA and comprises eIF4G, eIF4E, and eIF4A. eIF4G is a scaffolding protein that positions eIF4F for heterotrimeric engagement and initiation factor-RNA binding, eIF4E binds the m⁷G mRNA cap, and eIF4A is the enzymatic driver of the complex. eIF4A is an ATP-dependent DEAD-box RNA helicase that binds and unwinds secondary structure in the 5′-untranslated region (UTR) of mature mRNA, allowing the ribosome access to translate downstream genes. Mammalian cells have three eIF4A isoforms. eIF4AI (referred to as eIF4A throughout) and eIF4AII are 90% homologous, located in the cytosol, and can both incorporate into the eIF4F complex. However, eIF4AI is ten times more abundant than eIF4AII in growing cells, and eIF4AII is unable to rescue inhibition of translation and cellular proliferation upon eIF4AI suppression, suggesting that eIF4AI and eIF4AII are functionally distinct in vivo. eIF4AIII is a component of the exon-junction complex in the nucleus and is not involved in protein synthesis.

mRNAs are translated at different rates due to heterogeneity of secondary structure in their 5′-UTRs, and eIF4A discriminates between mRNAs based on 5′-UTR complexity. mRNAs with increased structure show increased dependence on eIF4A for translation, whereas initiation of simple, unstructured mRNAs is less reliant on eIF4A. Overexpression of eIF4A elicits small changes in overall protein synthesis rates, but enables a large, disproportionate, and specific increase in translation of a subset of mRNAs. mRNA identification studies reveal that the production of housekeeping proteins such as beta-actin and GAPDH is not altered by changes in eIF4A levels. Conversely, the translation of mRNAs encoding proteins involved in sustaining the hallmarks of cancer such as MYC, MCL1, BCL-xL, and cyclin-D1, which harbor long, highly-structured 5′-UTRs, is notably impacted by fluctuating eIF4A levels. Despite the essential nature of eIF4A in all cells, the increased dependency of oncoprotein production on eIF4A along with increased protein synthesis demands in cancer cells creates a therapeutic window in which cancer cells are more dependent on eIF4A than healthy cells. As a result, effects of eIF4A inhibition are biased toward cancer cells, which makes eIF4A an intriguing target for cancer therapy.

Many cancer drugs target signaling proteins such as kinases, and although these drugs have been useful in cancer treatment, they commonly have short-lived clinical responses. This is a result of cancer cells bypassing many of these proteins through parallel or redundant signaling. This leads to resistance, as inhibition of one point in a pathway can easily be circumvented by upregulation of other pro-oncogenic factors. eIF4A, however, is a convergence point for numerous oncogenic pathways, including Ras-Raf-ERK, EGFR, and PI3K-TOR. Therapies that target this junction for numerous different oncogenic pathways may overcome problems that hamper current cancer treatments such as tumor heterogeneity and inhibitor resistance.

Past in vitro screening for inhibitors of cap-dependent translation initiation using Krebs-2 translation extracts yielded natural products that all work by interfering with eIF4A. These compounds include hippuristanol, pateamine A (PatA), and silvestrol (a rocaglate or “flavagline”). Flavagline analogs are widely considered the most promising eIF4A inhibitors in the field, as eFT226 (Zotatifin), a rocaglamide-inspired inhibitor, has entered clinical trials for solid tumors (NCT04092673) as well as COVID-19 (NCT04632381). Previously, our group has also discovered natural product eIF4A inhibitors, including elatol, which is believed to bind eIF4A with 2:1 stoichiometry and shows potent eIF4A inhibition in cells. Common weaknesses of natural product inhibitors are they can be difficult to work with due to limited supply, and medicinal chemistry can be challenging due to synthetic inaccessibility.

Herein, the present invention features a novel eIF4A inhibitor with a unique mechanism of action and a novel binding pocket. In addition, the eIF4A inhibitor is a synthetically tractable small molecule allowing for further optimization.

### BRIEF SUMMARY OF THE INVENTION

It is an objective of the present invention to provide compositions and methods that allow for the inhibition of the eIF4A enzyme, as specified in the independent claims.

Embodiments of the invention are given in the dependent claims. Embodiments of the present invention can be freely combined with each other if they are not mutually exclusive.

In one embodiment, the present invention features an eIF4A inhibitor having a formula according to Formula I:

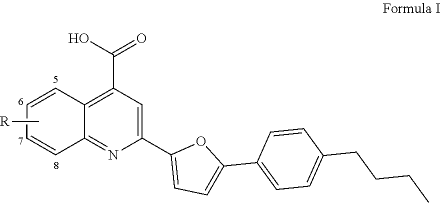

In another embodiment, the present invention features an eIF4A inhibitor of claim 1 having a formula according to Formula II:

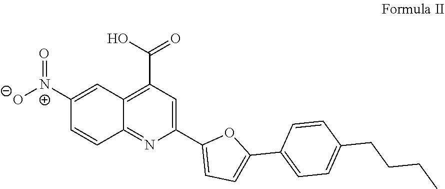 [SMILES: CCCCc1ccc(-c2ccc(-c3cc(C(=O)O)c4cc([N+](=O)[O-])ccc4n3)o2)cc1]

In yet another embodiment, the present invention features an eIF4A inhibitor having a formula according to Formula III:

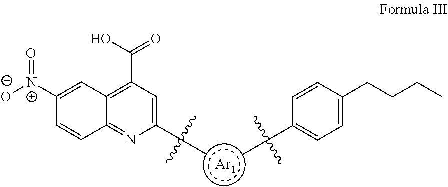 [SMILES: *c1cc(C(=O)O)c2cc([N+](=O)[O-])ccc2n1 ; *c1ccc(CCCC)cc1 ; "Ar1"]

In other embodiments, the present invention features eIF4A inhibitors having formulas according to Formula IV, Formula V, or Formula VI:

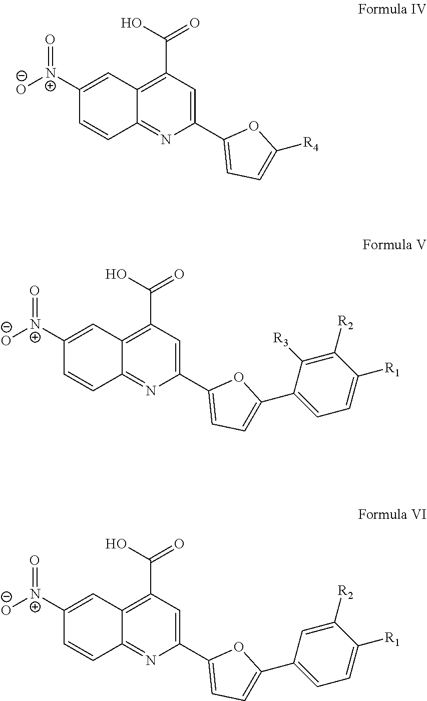

One of the unique and inventive technical features of the present invention is the novel mechanism of inhibiting eIF4A. Without wishing to limit the invention to any theory or mechanism, it is believed that the technical feature of the present invention advantageously provides for a novel mechanism for inhibiting the eIF4A enzyme. It was surprisingly found that the compounds described herein inhibit eIF4A with a novel mechanism, in which they perturb RNA binding, block the hydrolysis of ATP, and consequently, inhibit RNA helicase activity. Thus, the compounds of the present invention are RNA-competitive, ATP-uncompetitive eIF4A inhibitors that directly bind eIF4A and inhibit the protein.

None of the presently known prior references or work has the unique inventive technical feature of the present invention. For example, Rocaglates inhibit eIF4A by increasing the affinity of eIF4A for RNA and clamping eIF4A shut on RNA. PatA acts similarly, also stabilizing the eIF4A-RNA interaction. In addition, PatA stimulates eIF4A ATPase and helicase activities and inhibits the association of eIF4A and eIF4G, sequestering eIF4A from the eIF4F complex. The mechanism of elatol is not explicitly known, but its interaction with K82 of eIF4A suggests it may be ATP-competitive. Hippuristanol is the only other RNA-competitive inhibitor of eIF4A, but unlike the compounds of the present invention, it locks eIF4A closed and inhibits eIF4A unwinding activity by completely preventing RNA binding.

In addition, inhibition of eIF4A through this novel mechanism may result in downstream cellular consequences different from those resulting from clamping RNA onto eIF4A. This may allow for elucidation of additional pathways that are reliant on eIF4A for translation of protein. The compounds of the present invention may also occupy a novel binding pocket within the eIF4A RNA-binding groove. Rocaglates bind a small cavity that is encompassed by the N-terminal domain of eIF4A and polypurine RNA. Desmethyl pateamine A (DMPatA), a PatA analog, occupies largely the same binding site as rocaglates, forming major interactions with RNA and the N-terminal domain of eIF4A. However, unlike rocaglates, DMPatA also extends across the RNA to form minor interactions with the C-terminal domain of eIF4A, and is able to clamp onto both polypurine and polypyrimidine RNA. Elatol binds eIF4A in a 2:1 stoichiometric fashion, with both elatol molecules predicted to reside near the eIF4A ATP pocket. Hippuristanol, although it shares an RNA-competitive mechanism with the compounds of the present invention, binds exclusively to the C-terminal domain of eIF4A.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 shows a table of chemical structures of eIF4A inhibitors of the present invention and results from the RNA-stimulated malachite green ATPase assay.

FIG. 2 shows a table of chemical structures with different substitutions at the central ring of the inhibitor scaffold and their results from the RNA-stimulated malachite green ATPase assay.

FIG. 3 shows a table of chemical structures of eIF4A inhibitors containing substituted phenyl groups and results from the RNA-stimulated malachite green ATPase assay.

FIG. 4 shows a table of additional chemical structures of eIF4A inhibitors with substituted phenyl groups and results from the RNA-stimulated malachite green ATPase assay.

FIGS. 5A-5E show the discovery and initial biochemical testing of compound 1.

FIG. 5A shows RNA-accelerated malachite green ATPase assay screening data. Each black dot represents one compound. Green line indicates 3 standard deviations above the mean, which was the threshold used for hit determination (18 compounds). Initial hit 1 is circled in red.

FIG. 5B shows the chemical structure of compound 1.

FIG. 5C shows the dose response of compound 1 against eIF4A. IC50=26.6±2.46 μM. n=4.

FIG. 5D shows the kinetic testing of compound 1 using the malachite green assay. ATP titration (left) used 250 μg/mL RNA. Data were fit using unbiased mixed-model inhibition in Prism 6.0. Alpha value for ATP is 0.424, indicating an ATP-uncompetitive inhibitor. Kᵢₐₚₚ for ATP is 10.3 μM. RNA titration (right) used 250 μM ATP. Alpha value for RNA is 4.53×10¹³ indicating an RNA-competitive inhibitor. Kᵢₐₚₚ for RNA is 9.59 μM. Data shown are an average of 3 independent experiments.

FIG. 5E shows PDB: 5ZC9 on the left. RNA is shown as a red cartoon. AMP-PNP is shown as purple sticks. Compound 1 (cyan) docks to the RNA pocket of eIF4A (center). Zoomed surface view of the binding pocket is shown on the right.

FIGS. 6A-6D show mutagenesis studies that support docking pose of compound 28 binding to eIF4A RNA groove.

FIG. 6A shows a surface view of compound 28 in the RNA groove of eIF4A. (PDB: 5ZC9).

FIG. 6B shows compound 28 docked to eIF4A with close residues labeled and shown as pink sticks. Potential interactions between the quinoline side of compound 28 and the pocket are shown as orange dashes (right).

FIG. 6C shows a 2D view of the right panel of FIG. 6B. Distances indicated are Angstroms.

FIG. 6D shows potency of compound 28 against eIF4A mutants in the malachite green ATPase assay. Amino acid substitution is indicated under each bar. * Indicates IC50>50 μM.

FIG. 7A shows that compounds 1 and 28, but not compound 29, decrease BJAB cell viability. Compounds 1, 28, and 29 were tested using a CellTiter-Glo Luminescent Cell Viability assay in a BJAB Burkitt lymphoma cell line, n=4.

FIG. 7B shows that compound 28 inhibits cap-dependent translation. Top: Schematic representation of pSP/(CAG)₃₃/FF/HCV/Ren·pA₅₁ bicistronic reporter. Bottom: Effect of compound 28 on cap-dependent and HCV IRES-mediated translation in rabbit reticulocyte lysate programmed with pSP/(CAG)₃₃/FF/HCV/Ren·pA₅₁ vector. Luciferase luminescence values are normalized to signal obtained in control experiments with 1% DMSO. Cycloheximide (CHX) dose was 600 μM. Silvestrol dose was 100 nM. * Indicates a statistically significant difference between Renilla and firefly luciferase signal in a t test. p<0.002. n=3.

FIG. 7C shows that compound 28 engages eIF4A in cells. CETSA dose-response stabilization of eIF4A by compound 28 and silvestrol (100 nM) in A549 cells. Values represent western blot band intensities of eIF4A measured using densitometry. Intensities are normalized to GAPDH and subtracted from intensity of DMSO-treated sample.

FIGS. 8A-8D show that compound 28 is an RNA-competitive, ATP-uncompetitive eIF4A inhibitor.

FIG. 8A shows kinetic testing of compound 28 using the malachite green assay. ATP titration (left) was done with 250 μg/mL RNA. Data were fit using unbiased mixed-model inhibition in Prism 6.0. Alpha value for ATP is 0.514, indicating an ATP-uncompetitive inhibitor. Kᵢₐₚₚ for ATP=4.79 μM. RNA titration (right) was done with 250 μM ATP. Alpha value for RNA is 4.52×10²¹ indicating an RNA-competitive inhibitor. Kᵢₐₚₚ for RNA=4.27 μM. Data shown are an average of 3 independent experiments.

FIG. 8B shows the FP assay with MANT-ATP as the fluorophore. PEL=Polarized Excitation Light.

FIG. 8C shows the FP assay with FAM-labeled RNA as the fluorophore (left). compound 28 IC50=58.3±2.5 μM. Compound 1 IC50=161±9.8 μM (center). The center experiment utilized FAM-A(CAA)5 RNA. The right experiment utilized two different RNAs.

FIG. 8D shows the thermal shift assay with compound 28 and eIF4A. Error bars are too small to see, n=3.

FIG. 9 shows that compounds 1 and 28 inhibit eIF4A duplex unwinding. Cartoon of duplex used in the unwinding assay (top-right) with Cy3-labeled RNA (orange) and DNA loading strand (black). Increasing concentrations of compounds 1 and 28 were added to unwinding reactions containing duplex, eIF4A, and ATP (left). Products of unwinding reactions were resolved on native polyacrylamide gels. Cy3 signal on gels was quantified by band densitometry. Percent inhibition for each reaction was calculated using (Cy3 duplex signal/total Cy3 signal), n=3.

FIG. 10 shows a model of mechanism of eIF4A inhibition by compound 28. Under normal conditions (top), eIF4A can bind ATP (purple) and RNA (red), unwind the RNA, hydrolyze the ATP, and release its substrates. Upon introduction of compound 28 (bottom), eIF4A can bind ATP and weakly bind RNA, but in a conformation that hinders its ability to hydrolyze ATP and unwind RNA.

FIG. 11A shows a cartoon of the malachite green assay used in these studies.

FIG. 11B shows the presence of 250 μg/mL Yeast RNA in the malachite green assay accelerates eIF4A ATPase activity without increasing background.

FIG. 11C shows the Z-Factor of each 384 well plate used for singlicate screening, triplicate testing, and initial dose responses. Average Z Factor of every plate was 0.74.

FIG. 12 shows a table of DSF of eIF4A and mutants.

FIG. 13 shows that compounds 1 and 28 inhibit eIF4A duplex unwinding. The products of unwinding reactions were analyzed by electrophoresis on 20% native polyacrylamide (37.5:1 acrylamide:bisacrylamide) gels for 2 h at 150 V at 4° C. in 1×TBE buffer. All gel lanes contained single-stranded Cy3-labeled RNA. The number on the left indicates the compound used. DS=double stranded Cy3-duplex. SS=single stranded Cy3RNA. One representative gel is shown for each compound. n=3.

FIG. 14 shows that the furan moiety of compound 28 may form a hydrogen bond with T109. Compound 28 docked to eIF4A (PDB: 5ZC9). T109 is labeled and shown as pink sticks. Potential hydrogen bond between 28 and T109 is shown as orange dashes. Distance indicated is Angstroms.

FIG. 15 shows that decrease in BJAB cell viability tracks eIF4A ATPase inhibition. Compounds 9, 24, 25, 46, and 47 were tested using a CellTiter-Glo Luminescent Cell Viability assay in a BJAB Burkitt lymphoma cell line. n=4.

FIG. 16 shows a western blot of isothermal dose-response of 28 in A549 cells. All dose response CETSAs were conducted using previously determined in-cell eIF4A melting temperature of 55° C. Silvestrol treatment was 100 nM.

### DETAILED DESCRIPTION OF THE INVENTION

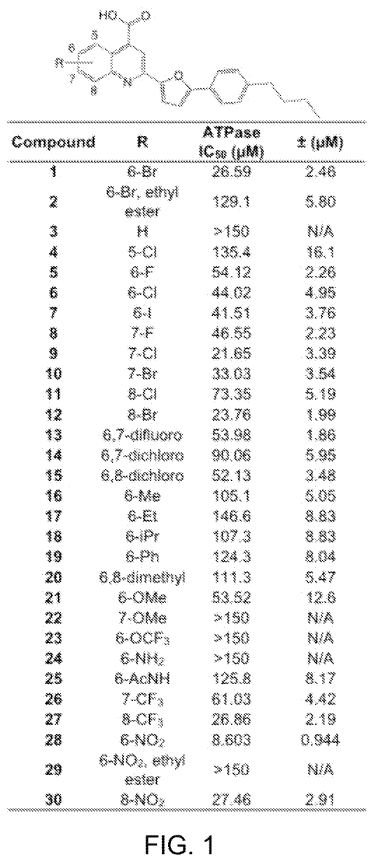

Referring now to FIG. 1, the present invention features an eIF4A inhibitor having a formula according to Formula I:

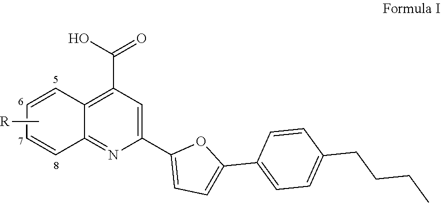

In some embodiments, R may be, but it not limited to, H, alkyl, substituted alkyl, aryl, substituted aryl, heteroaryl, substituted heteroaryl, amino, alkoxy, nitro, halo, or a combination thereof.

In another embodiment, the eIF4A inhibitor may have a formula according to Formula II:

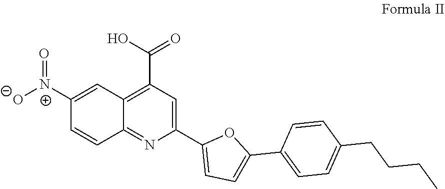 [SMILES: CCCCc1ccc(-c2ccc(-c3cc(C(=O)O)c4cc([N+](=O)[O-])ccc4n3)o2)cc1]

In yet another embodiment, the present invention features an eIF4A inhibitor having a formula according to Formula III:

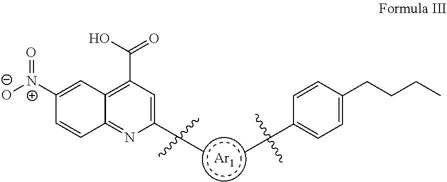 [SMILES: *c1cc(C(=O)O)c2cc([N+](=O)[O-])ccc2n1 ; *c1ccc(CCCC)cc1 ; "Ar1"]

In some embodiments, Ar₁ may be, but is not limited to, aryl, substituted aryl, heteroaryl, or substituted heteroaryl.

In other embodiments, the present invention features an eIF4A inhibitor having a formula according to Formula IV:

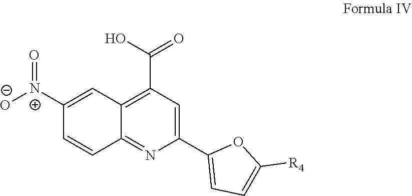

In some embodiments, R₄ may be, but is not limited to, aryl, substituted aryl, heteroaryl, or substituted aryl.

In further embodiments, the eIF4A inhibitor may have a formula according to Formula V or Formula VI:

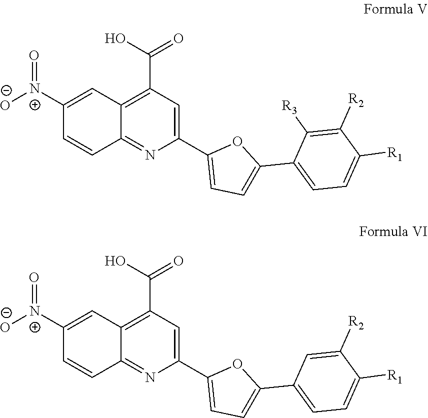

In some embodiments, R₁, R₂, and R₃ may be each independently H, alkyl, substituted alkyl, alkoxy, aryl, or amino. Without wishing to limit the present invention to any mechanism or theory, each of the eIF4A inhibitors described herein may occupy a binding pocket of an eIF4A RNA-binding groove, thereby perturbing RNA binding to eIF4A, blocking ATP hydrolysis, and inhibiting eIF4A from unwinding RNA.

In further embodiments, the eIF4A inhibitor may have another formula which allows the inhibitor to occupy the same binding pocket of eIF4A as the inhibitor of Formula II. As a non-limiting example, the eIF4A inhibitor may be configured to bind or interact with one or more of R110, T158, and R311 of eIF4A. As another non-limiting example, the eIF4A inhibitor may be configured to bind or interact with all three of R110, T158, and R311 of eIF4A The eIF4A inhibitor may be an ATP uncompetitive and RNA competitive inhibitor. The eIF4A inhibitor may be configured to inhibit eIF4A unwinding while incompletely inhibiting RNA binding.

The present invention features a method for inhibiting eIF4A activity in a cell. The method may comprise contacting the cell with an eIF4A inhibitor. The eIF4A inhibitor may have a formula according to Formula I, Formula II, Formula III, Formula IV, Formula V, Formula VI, or another formula. Without wishing to limit the present invention to any theory or mechanism, the eIF4A inhibitor may occupy a binding pocket of an eIF4A RNA-binding groove, thereby perturbing RNA binding to eIF4A, blocking ATP hydrolysis, and inhibiting eIF4A from unwinding RNA. As a non-limiting example, the eIF4A inhibitor may be configured to occupy the same binding pocket of eIF4A as the inhibitor of Formula II. As another non-limiting example, the eIF4A inhibitor may be configured to bind or interact with one or more of R110, T158, and R311 of eIF4A.

### Example

The following is a non-limiting example of the present invention. It is to be understood that said example is not intended to limit the present invention in any way. Equivalents or substitutes are within the scope of the present invention.

### Results

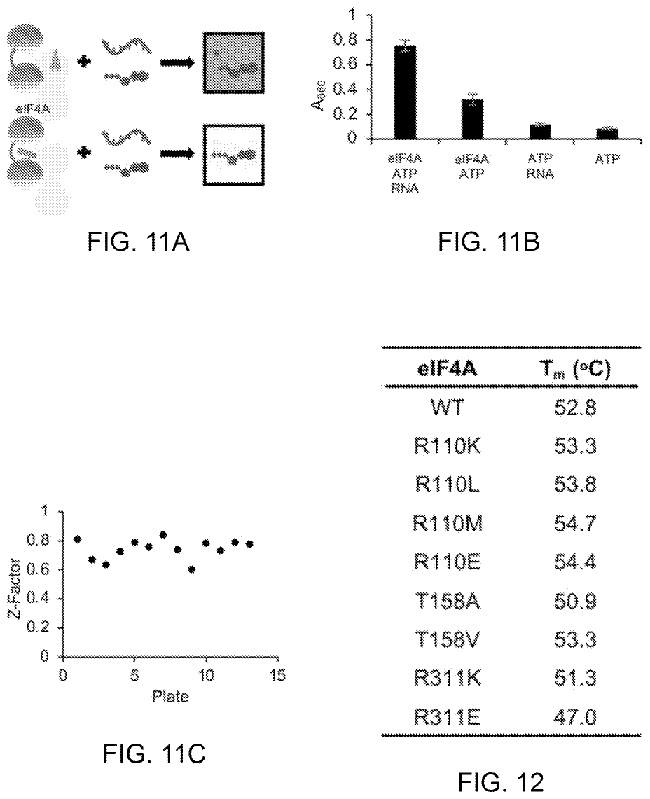

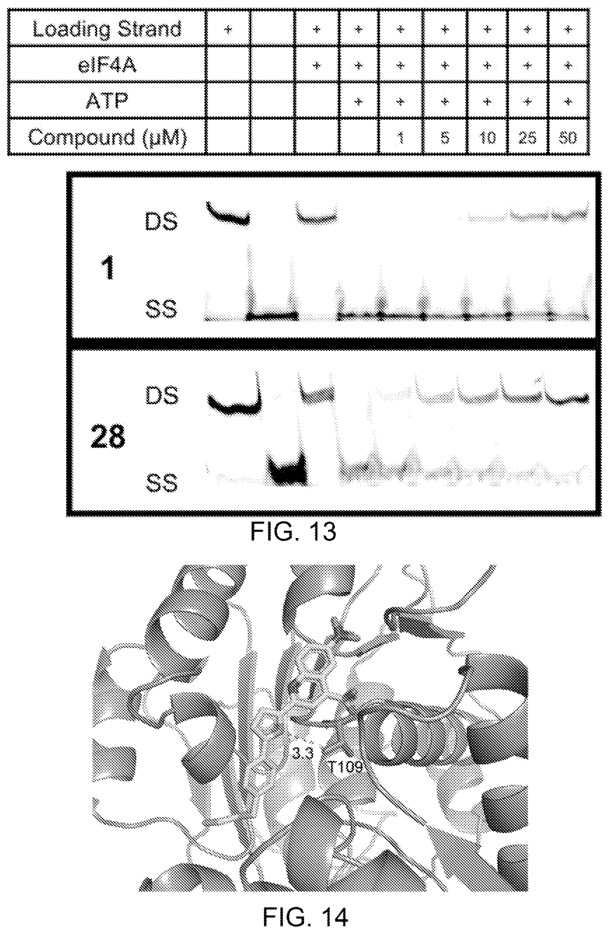

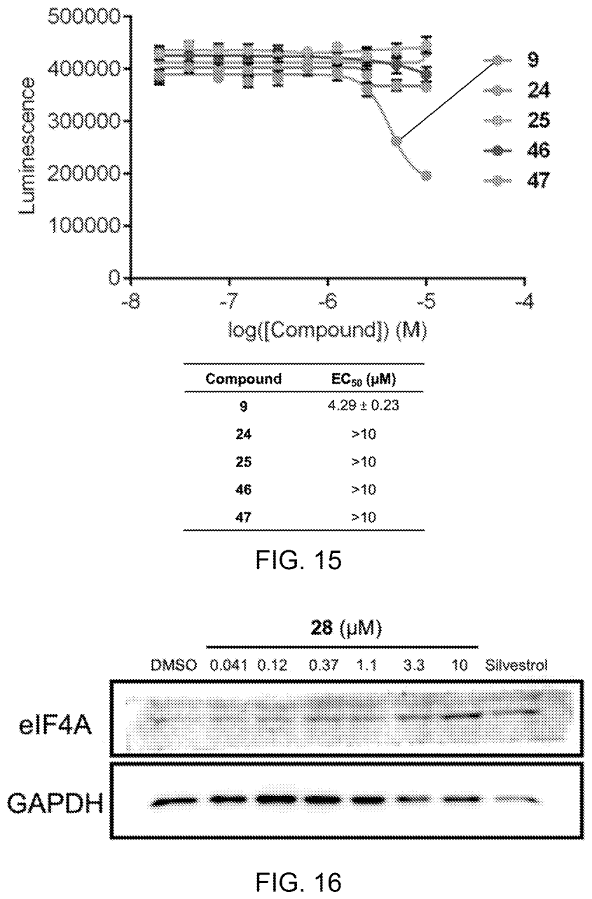

Human eIF4A protein was produced recombinantly and a greater than 100,000-compound screen for inhibitors was completed using a malachite green ATPase assay (FIG. 11A). Surprisingly, the only hits identified were not potent and could not be validated in triplicate and dose response studies (data not shown). A stable mixture of yeast RNAs was added in an effort to improve this system and create a more biologically relevant eIF4A assay. eIF4A ATPase activity was accelerated without a statistically significant increase in the background signal in the presence of this RNA (FIG. 11B), and the new conditions gave excellent statistical parameters in a 384-well plate format (FIG. 11C).

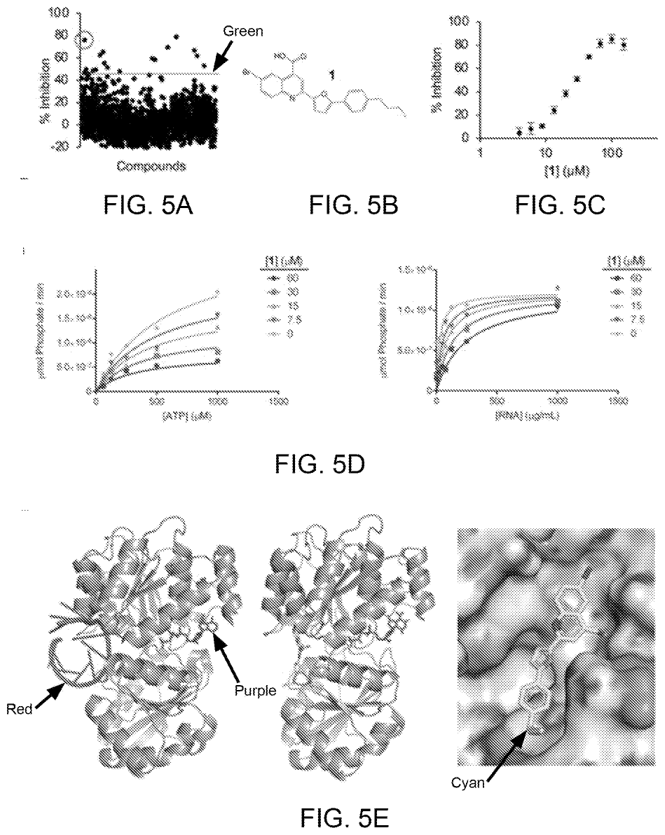

This unique RNA-stimulated malachite green ATPase assay was repeated in a pilot screen for inhibitors of eIF4A. Eighteen hits were identified in this screen (FIG. 5A), the most potent being a 2-(5-(4-butylphenyl)furan-2-yl)quinoline-4-carboxylic acid, compound 1 (FIG. 5B), with an IC₅₀ of 26.59±2.46 μM (FIG. 5C). This molecule passed Lipinski and PAINS filters with zero violations according to SwissADME, and was evaluated in kinetics assays to study its mechanism of inhibition. The expectation was that compound 1 would be an ATP-competitive inhibitor, due to its ability to inhibit the ATPase activity of eIF4A. However, surprisingly, kinetic testing showed that compound 1 was uncompetitive with respect to ATP (Kᵢₐₚₚ=10.3 μM), as Km and Vmax of eIF4A both decreased upon addition of compound 1 (FIG. 5D, left). Instead, compound 1 was found to be RNA-competitive (Kᵢₐₚₚ=9.59 μM), as the Km of eIF4A decreased and Vmax was constant upon treatment with compound 1 (FIG. 5D, right).

Molecular docking was performed to predict the binding site of 1, and as expected for a RNA-competitive inhibitor, it docked to the RNA-binding groove of eIF4A (FIG. 5E). This predicted binding pocket engages both RecA-like domains of eIF4A and has never been accessed before by a small molecule inhibitor. Visualization of the probable binding mode of compound 1 suggested that modification of the inhibitor could increase its favorable molecular interactions with the binding pocket and enhance its potency (FIG. 5E, right). This observation, along with the uniqueness of the binding location and mechanism of inhibition of 1, encouraged us to synthesize a small library of analogs to expand this class of inhibitors.

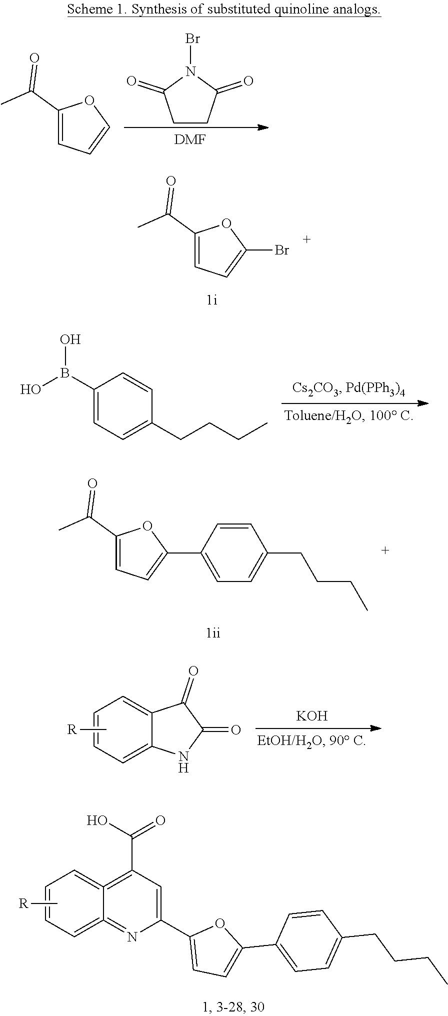

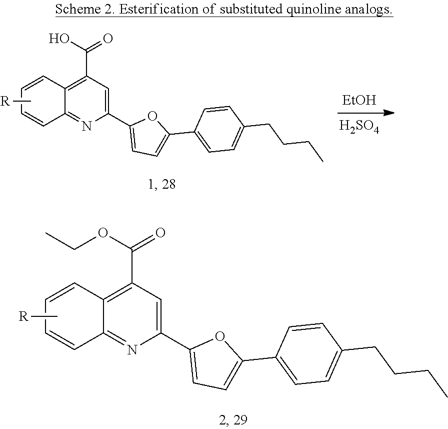

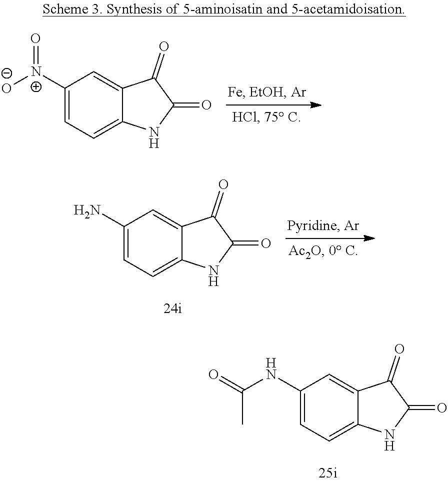

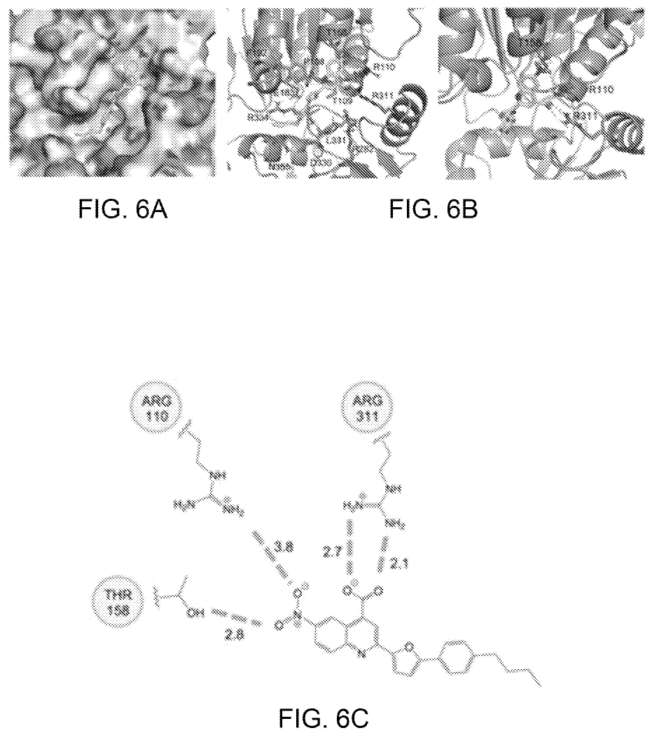

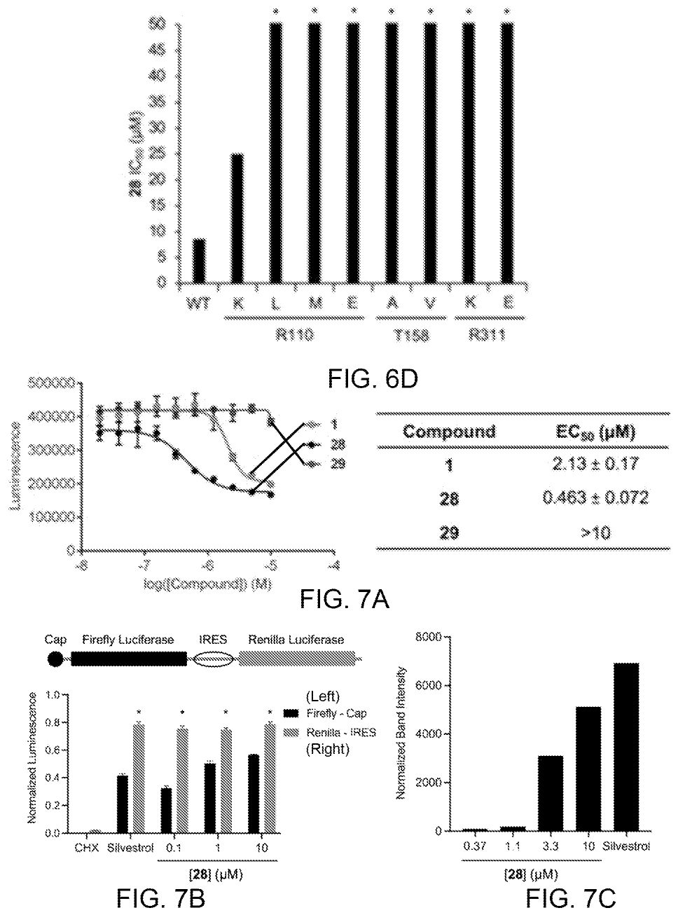

Synthesis of compound 1 analogs (Scheme 1) began with bromination of 2-acetylfuran with N-bromosuccinimide to yield 1i. The brominated product was utilized in a Suzuki coupling with 4-butylphenylboronic acid to produce 1ii. This intermediate was stockpiled and employed in Pfitzinger reactions with substituted isatins to afford 2-(5-(4-butylphenyl)furan-2-yl)quinoline-4-carboxylic acids with varied quinoline substitutions in 21%-39% yield over three steps. The carboxylic acids could also easily be esterified upon addition of sulfuric acid in ethanol (Scheme 2). Each molecule was tested in the RNA-stimulated malachite green ATPase assay (FIG. 1). Compound 2, the 1-ethyl ester, was about 5-fold less potent than compound 1, which suggested that the carboxylic acid played an important role in eIF4A inhibition. This was expected, as our docking data indicated the acid moiety formed a salt bridge with R311 of eIF4A (FIG. 6).

Compound 3, with no bromine, was also much less potent than compound 1, indicating that a quinoline substitution was necessary for inhibition of eIF4A. Subsequent analogs explored the effect of various halogen substitutions on each position of the quinoline ring. The 5-position of the quinoline was immediately ruled out as 4 showed a large drop in potency. Functionalizing the 6-position of the quinoline with a chlorine (6) or iodine (7) atom revealed moderate losses of potency compared to the bromine substituent, possibly due to unfavorable atomic size. Analogs with fluorine (8), chlorine (9), and bromine (10) substitutions to the 7-position of the quinoline displayed moderate potency, with 9 performing the best in the ATPase assay of any inhibitor produced. At the 8-position of the quinoline, the chlorine substitution (11) was less potent than bromine (12). This group of molecules was unable to produce a lead with a potency lower than 20 μM in the ATPase assay, so a few di-substituted halogen analogs were synthesized in an attempt to increase potency of the series. However, 6,7 and 6,8 substituted analogs 13-15 did not afford increased potency.

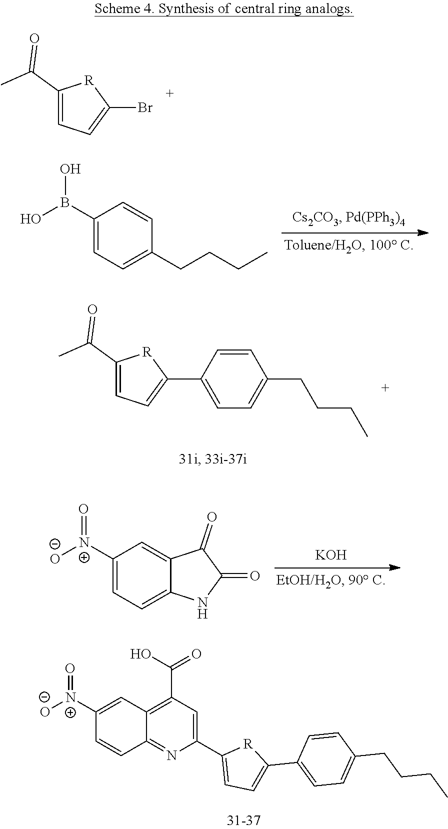

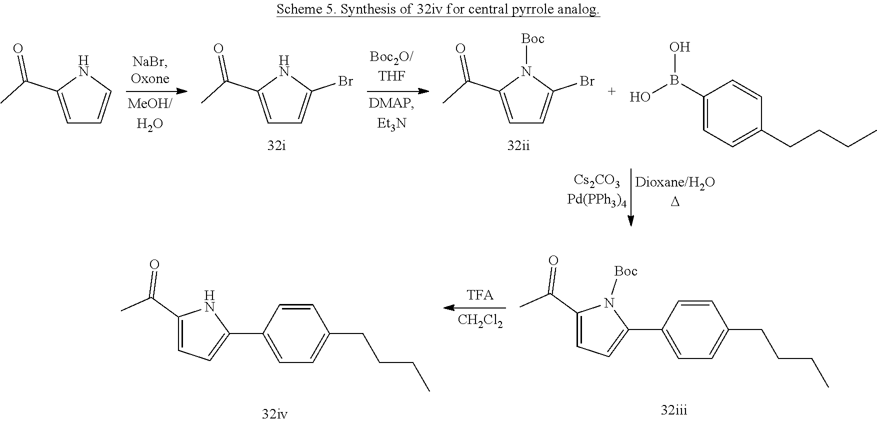

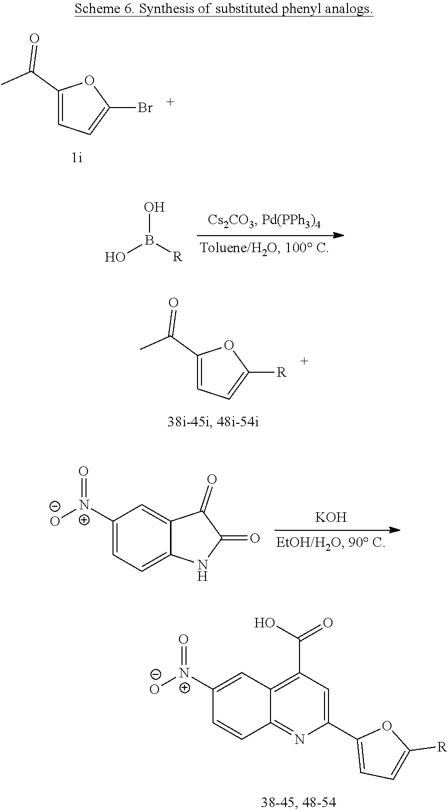

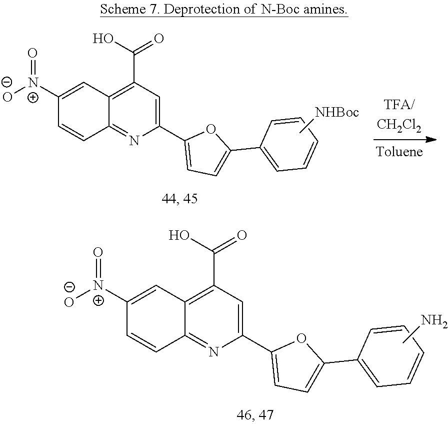

A shift to production of hydrocarbon-substituted quinolines at position 6 with compounds 16-19 revealed that the size of the substitution may not play as prominent of a role in conferring potency of eIF4A inhibition in this binding pocket as originally expected. Despite a drastic gain in size from compounds 16-19, potency stayed low and relatively constant. This trend was consistent with dimethyl-substituted 20. This suggested that potent interactions between the quinoline moiety and the eIF4A binding pocket were driven by electronic interactions and not purely by the size of the substitution. The next molecules produced featured electron-donating groups at the 6 and 7-positions of the quinoline. 6-methoxy (21) was much more potent than 7-methoxy (22), but neither were more potent than earlier molecules in the series. In addition, 6-trifluoromethoxy (23) was much less potent than compound 21.

The next two analogs produced with electron-donating group substitutions on the quinoline were 6-amino (24) and 6-acetamido (25) (Scheme 3), which also showed major losses in potency. Failure to increase potency with electron-donating substitutions on the quinoline led to production of analogs with electron-withdrawing group substitutions. 7-trifluoromethyl (26) and 8-trifluoromethyl (27) performed better in the ATPase assay than molecules with electron-donating substitutions, and compound 27 faired about two-fold better than compound 26. Consequently, a strongly electron-withdrawing group was placed at the 6-position of the quinoline to produce compound 28, which had an IC₅₀ of 8.603±0.944 μM in the eIF4A ATPase assay. This was over two-fold more potent than any other molecule previously produced in the series. Immediately, compound 28 was esterified to compound 29, which lost all eIF4A potency, further reinforcing the importance of the carboxylic acid moiety to eIF4A ATPase inhibition by this class of molecules (FIG. 6). Finally, due to the relative success of compound 27 with an electron withdrawing group at position 8 on the quinoline, compound 30 was synthesized with a nitro group at this position. Compound 30 was over three-fold less potent than compound 28. compound 28, like compound 1, passed Lipinski and PAINS tests with no violations according to SwissADME, predicting that compound 28 has a favorable physicochemical profile.

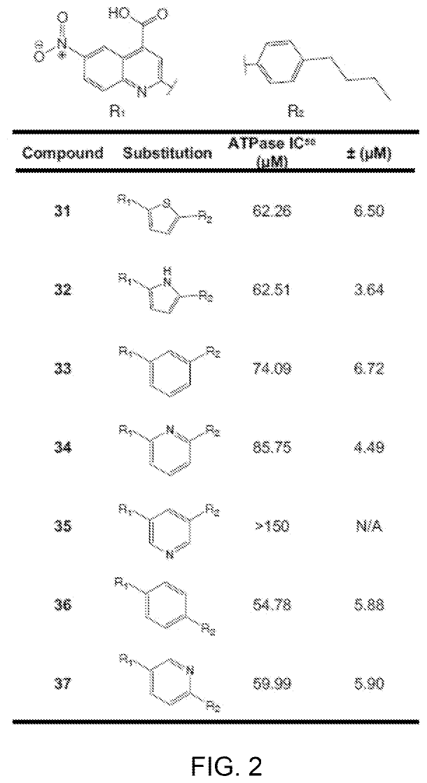

Additional analogs of 28 were synthesized to probe structure-activity relationships between different moieties of 28 and the eIF4A binding pocket. Initially, the nitroquinoline and butylphenyl groups were held constant, while the furan was substituted for different five and six-membered rings. To produce these analogs, brominated, acetylated homocyclic and heterocyclic rings were utilized in Suzuki couplings with 4-butylphenylboronic acid, and the resulting intermediates were employed in Pfitzinger reactions with 5-nitroquinoline to result in central ring analogs 31-37 (Scheme 4). The sequence of the synthesis was unchanged from the derivatization of the quinoline, as performing the Pfitzinger reaction as the last step produced pure compounds in high yields. The advanced pyrrole intermediate 32iv was produced differently than the other molecules, as the amine of a pyrrole must be protected before it is employed in a Suzuki coupling to prevent debromination of the starting material.30 2-acetylpyrrole was selectively brominated at the 5-position with sodium bromide and Oxone, and the pyrrole amine was protected with a tert-butoxycarbonyl (Boc) protecting group. The protected pyrrole was employed in a Suzuki coupling to attach the butylphenyl moiety and the pyrrole was deprotected through removal of Boc to produce 32iv (Scheme 5), which was utilized in a Pfitzinger reaction with 5-nitroquinoline to produce the pyrrole analog 32. Each of these analogs were tested in the eIF4A ATPase assay (FIG. 2).

Replacement of furan with 5-membered heterocycles thiophene (31) and pyrrole (32) led to a large drop in potency in the ATPase assay. The next three molecules produced (33-35) were six-membered rings with the butylphenyl group positioned meta to the quinoline. Compounds 33 and 34 experienced a drop in potency in the ATPase assay and 35 lost potency altogether. Analogs 36 and 37, with the quinoline and butylphenyl groups positioned para to each other, were also much less potent than 28 in the ATPase assay.

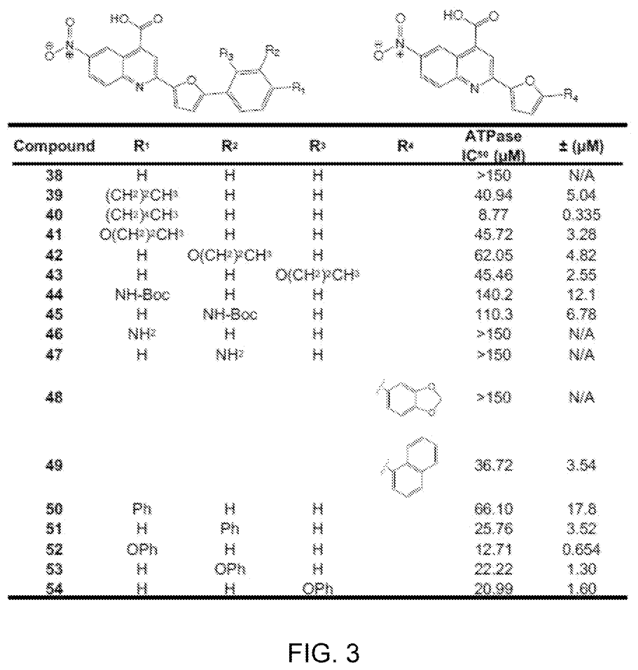

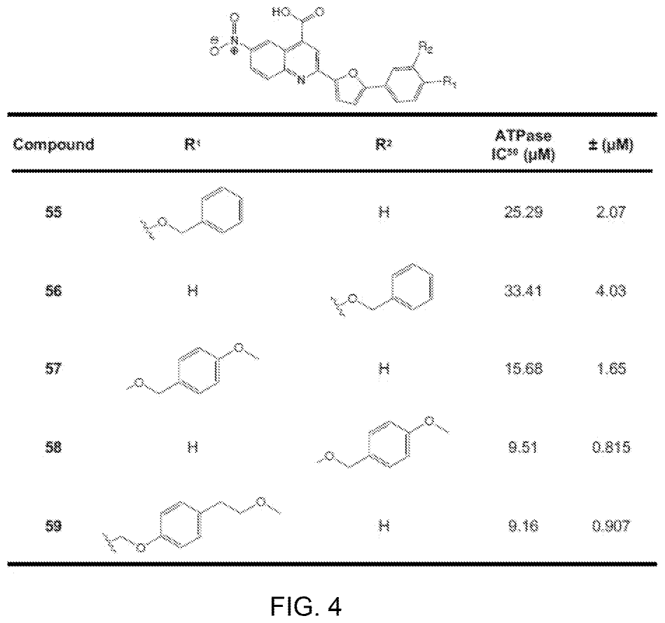

Finally, analogs with changes to the butylphenyl moiety were produced leaving the nitroquinoline and furan rings unchanged. These compounds were produced with the same general synthetic scheme as the central ring analogs, and derivatization resulted from employing different boronic acid species in the Suzuki coupling (Scheme 6). Compounds 38-45 and 48-54 were produced in this manner, and 46 and 47 were the result of Boc deprotection of 44 and 45, respectively (Scheme 7). Each compound was tested against eIF4A in the malachite green ATPase assay (FIG. 3).

The first substituted phenyl analog synthesized and tested was compound 38, which had no butyl tail. This molecule experienced a drastic loss in potency, indicating that substitution to the phenyl ring was important to eIF4A inhibition by this class of molecules. Next, phenyl rings substituted with para-hydrocarbon chains of different lengths were tested. Compounds 39 and 40 featured propyl and pentyl chains, respectively, which are one carbon shorter and longer than the butyl chain of 28. Shortening the chain did not improve potency, as compound 39 was about 5-fold less potent than 28, and lengthening the chain also did not improve potency, as 40 was no more potent than 28. With optimal chain length established, different positions of the chain were investigated with propylether phenyl substitutions. Compounds 41-43 all experienced drops in potency in the ATPase assay. Boc substitutions to the phenyl ring were also investigated with compounds 44 and 45 before being deprotected to amines 46 and 47. All four of these compounds showed low potency in the ATPase assay.

Various ring species were synthesized to explore the necessity for a phenyl at this position. Compound 48, with the benzo[1,3]dioxole substitution to the furan, lost all potency in the ATPase assay, while the 1-naphthyl-substituted compound 49 was about four-fold less potent than compound 28. Finally, phenyl and phenoxy substitutions at varied positions of the phenyl moiety were explored. Phenyl-substituted compounds 50 and 51 were less potent than compound 28 in the ATPase assay, with para-substituted 50 more potent than meta-substituted 51. Phenoxy substituted compounds 52-54 were generally more potent than 50 and 51, but not as potent as compound 28. Compound 28 was the most potent inhibitor of eIF4A ATPase activity, and it was carried forward for additional exploration into its mechanism of inhibition.

Molecular docking of compound 28 to eIF4A was performed to investigate the potential molecular interactions driving the potency of compound 28 for eIF4A. Unsurprisingly, compound 28 docked to the same pocket as compound 1 in the RNA-binding groove of eIF4A (FIG. 6A). The docking predicts several close contacts between compound 28 and the amino acids that form the binding pocket, but the main residues that appear to drive binding are R110, T158, and R311 (FIG. 6B). R110 and R311 are part of a string of positively charged amino acids that line the RNA-binding groove of eIF4A, allowing eIF4A to bind the negatively charged sugar-phosphate backbone of RNA. The negatively charged oxygen atoms of the nitro and acid groups are predicted to form salt bridges with R110 and R311, respectively, which would disrupt the interaction between eIF4A and RNA (FIG. 6C).

Furthermore, T158 is predicted to form a hydrogen bond to the carbonyl group of the 6-nitro quinoline substitution of compound 28. The ability of the 6-nitro group to form two strong interactions with the binding pocket of eIF4A is the most likely explanation for why this substitution afforded 28 such a large gain in potency for compound 28 compared to other analogs in the inhibitor series. Mutagenesis and recombinant production of mutant eIF4A protein was performed to confirm the docking pose and predicted interactions between compound 28 and its eIF4A binding pocket (FIG. 6D). All eIF4A mutants produced were determined to be thermodynamically stable by differential scanning fluorimetry (DSF), as each mutant had a melting temperature indicative of a properly folded protein (FIG. 12). Compound 28 remained moderately potent for the R110K mutant, which retains its positive charge, while R110L, R110M, and R110E mutants lost all potency, supporting the presence of an ionic interaction between compound 28 and R110. Compound 28 also lost all potency for T158A and T158V eIF4A mutants, which do not have a hydrogen bond donor to contribute an interaction with compound 28.

These results strongly support the presence of a hydrogen bond between T158 and compound 28. Furthermore, eIF4A R311K and R311E mutants were produced and tested with compound 28. As expected, compound 28 had no potency toward R311E, as the positively charged arginine is replaced with a negatively charged glutamic acid that repels the carboxylic acid of compound 28. However, compound 28 also had no potency for eIF4A R311K, even though it still maintains its positive charge. This may be attributed to the inability of this lysine to simultaneously provide hydrogen bond donation and ionic interactions to compound 28 due to geometrical constraints. The molecular docking also predicted that the furan of 28 may form a hydrogen bond with T109, and the butyl group may lie within a small hydrophobic groove within the binding pocket (FIG. 6A). Nevertheless, mutations to R110, T158, and R311 causing drastic losses in potency of compound 28 provided strong evidence that this was the correct binding pocket and pose for compound 28. This site, wedged between each RecA-like domain of eIF4A, is a novel binding pocket that has never been accessed by an eIF4A inhibitor.

Compounds 1 and compound 28 were evaluated in a CellTiter-Glo (CTG) Luminescence cell viability assay in BJAB Burkitt lymphoma cells that are known to be sensitive to eIF4A inhibition. This assay quantitates the number of living, metabolically active cells by measuring their ATP levels. Compound 1 decreased BJAB cell viability with an EC₅₀ of 2.13±0.17 μM (FIG. 7). Compound 28 also decreased viability, and like in the ATPase assay, compound 28 was three to four times more potent than 1, with an EC₅₀ of 0.46±0.07 μM. Compound 29, the 28-ethyl ester, is not potent in the ATPase assay, likely due to the removal of the interaction between the carboxylic acid and R311 of eIF4A. Compound 29 was tested in the BJAB viability assay and did not inhibit cellular viability, showing that structure-activity relationships of 28 and 29 correlate between the ATPase and cellular assays.

Additional analogs with varying potencies in the ATPase assay (9, 24, 25, 46, 47) were tested in the cell viability assay to investigate if ATPase inhibition tracked BJAB CTG inhibition and to ensure that cell viability was not decreasing due to general toxicity of the inhibitor scaffold. The potency of compound 9 in the ATPase assay (IC₅₀=21.65±3.39 μM) is similar to that of compound 1 (IC₅₀=26.59±2.46 μM). Both compounds also inhibited BJAB viability with similar EC₅₀ values (9 EC₅₀=4.29±0.23 μM, 1 EC₅₀=2.13±0.17 μM) (Figure s3). Compounds 24 and 25 retain the furan and butylphenyl moieties of 28, but have different quinoline substitutions, causing them to lose potency in the ATPase assay. These molecules do not decrease BJAB cellular viability. Furthermore, compounds 46 and 47 retain the nitroquinoline and furan moieties of 28, but feature different phenyl substitutions, causing them to lose potency in the ATPase assay. As expected, these molecules also do not inhibit BJAB cell viability. Minor substitutions to compound 28 that reduce potency in the ATPase assay also reduce potency in the BJAB CTG assay. The eIF4A ATPase assay inhibition data closely tracks the BJAB viability data, providing additional evidence that 28 decreases BJAB cell viability as a result of inhibiting eIF4A.

In vitro translation studies were performed to confirm the ability of 28 to inhibit cap-dependent translation in an environment with complete cellular translation machinery and further link inhibition of cellular viability by 28 to inhibition of eIF4A. A bicistronic luciferase reporter (pSP/(CAG)₃/FF/HCV/Ren-pA₅₁) (FIG. 7B, top) was programmed into rabbit reticulocyte lysates, cellular extracts capable of performing both cap-dependent and Hepatitis C virus (HCV) internal ribosomal entry site (IRES)-mediated translation. eIF4A is the enzymatic driver of cap-dependent translation and is not involved in translation through HCV IRES. Compounds that inhibit eIF4A should inhibit cap-dependent translation of firefly (FF) luciferase while leaving IRES-mediated translation of Renilla (Ren) luciferase relatively unaffected. The inhibitory activities of cycloheximide, silvestrol, and 28 were assessed in this in vitro translation assay (FIG. 7B, bottom). Cycloheximide, an inhibitor of translation elongation, inhibits both cap-dependent and IRES-mediated translation. Compound 28 and silvestrol both inhibit cap-dependent translation of firefly luciferase, but only show a modest effect on HCV IRES-dependent translation of Renilla luciferase. The ability of compound 28 to preferentially target cap-dependent translation in a similar manner as silvestrol provides promising evidence that 28 inhibits eIF4A and not other proteins in the general translational machinery.

A cellular thermal shift assay (CETSA) was performed to test the ability of 28 to bind and stabilize eIF4A in cells and connect its inhibition of cellular viability and cap-dependent translation to engagement of eIF4A. In CETSA, thermal stability of a protein is assessed by heating cells and then separating insoluble, aggregated, or denatured proteins from soluble proteins through supernatant/pellet fractionation. Levels of the protein of interest (eIF4A) remaining in the soluble fraction are then measured by western blot. The principle of CETSA is similar to differential scanning fluorimetry (DSF), in that thermal stability and solubility of a protein increases upon binding a ligand. Treatment of A549 cells with silvestrol increased thermal stability of eIF4A (FIG. 7C). Similarly, treatment with compound 28 caused a dose-dependent increase in thermal stability of eIF4A as more eIF4A appeared in the soluble fraction (FIG. 16). Taken together, compound 28 inhibits BJAB cell viability, inhibits cap-dependent translation, and engages eIF4A in cells, indicating that 28 confers its cellular potency through engagement and inhibition of eIF4A.

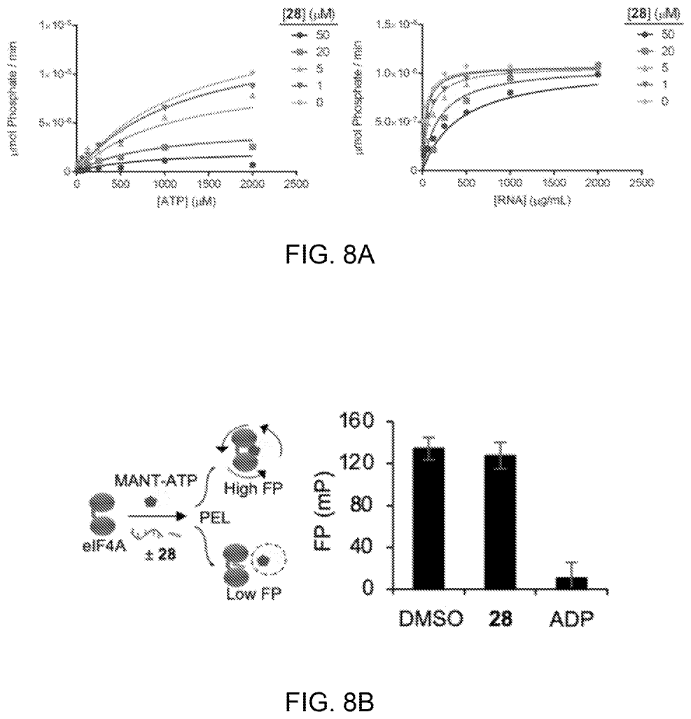

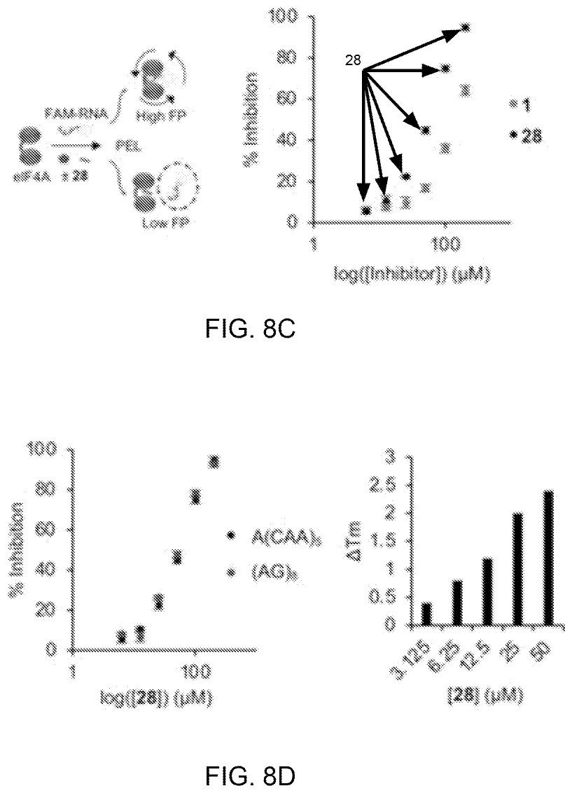

Additional biochemical testing was performed on compound 28 to investigate its mechanism of eIF4A inhibition. Initially, kinetics experiments were repeated with compound 28, and the results for compound 28 matched those of compound 1 (FIG. 5D, 4A). Upon addition of compound 28, Km and Vmax of eIF4A decreased with respect to ATP. With respect to RNA, eIF4A Km decreased and Vmax remained constant. Consequently, compound 28 is predicted to act as an RNA-competitive (Kᵢₐₚₚ=4.27 μM), ATP-uncompetitive (Kᵢₐₚₚ=4.79 μM) eIF4A inhibitor that binds to ATP-bound eIF4A in the RNA groove. Fluorescence polarization (FP) assays were utilized to confirm this mechanism of inhibition by testing the ability of compound 28 to compete fluorescent substrates off eIF4A. First, an FP assay was performed with MANT-ATP, a fluorescent, non-hydrolysable ATP analog, as the substrate. ADP, but not compound 28, was able to compete MANT-ATP out of its binding pocket (FIG. 8B), confirming that compound 28 is not an ATP-competitive inhibitor.

Next, an FP assay was performed with a fluorescent, eIF4A-binding RNA oligo (FAM-A(CAA)₅) as the fluorophore. At high concentrations, both compound 1 and compound 28 were able to compete the fluorescent RNA off of eIF4A, indicating that compound 28 is an RNA-competitive inhibitor (FIG. 8C, middle). However, the potency of these molecules in this assay were surprisingly low, indicating that compound 1 and compound 28 do not fully compete RNA out of the RNA-binding groove at concentrations in which the compounds inhibit eIF4A ATPase activity (discussed below). The IC₅₀ of compound 1 was 160.6±9.8 μM, and the IC₅₀ of compound 28 was 58.3±2.5 μM, meaning compound 28 was about three-fold more potent than 1, which was consistent with the other assays performed. This same assay was used to investigate if changing the identity of the RNA fluorophore affected the ability of compound 28 to compete the RNA off eIF4A. FAM-(AG)₈ was utilized alongside FAM-A(CAA)₅ and the IC₅₀ of compound 28 in this assay was consistent regardless of RNA identity (FIG. 8C, right). This is evidence that compound 28 inhibits the RNA-eIF4A interaction by binding eIF4A and not the RNA.

Further evidence of this was uncovered in a thermal shift assay (TSA) using DSF with eIF4A and compound 28. Compound 28 increased eIF4A stability, exhibited by a rise in eIF4A melting temperature (FIG. 8D) upon heating of the protein. Larger doses of compound 28 caused larger melting temperature (Tm) shifts in this assay, which argues that compound 28 is directly binding eIF4A. These data indicate that compound 28 is an RNA-competitive, ATP-uncompetitive eIF4A inhibitor that directly binds eIF4A and inhibits the protein through a novel mechanism.

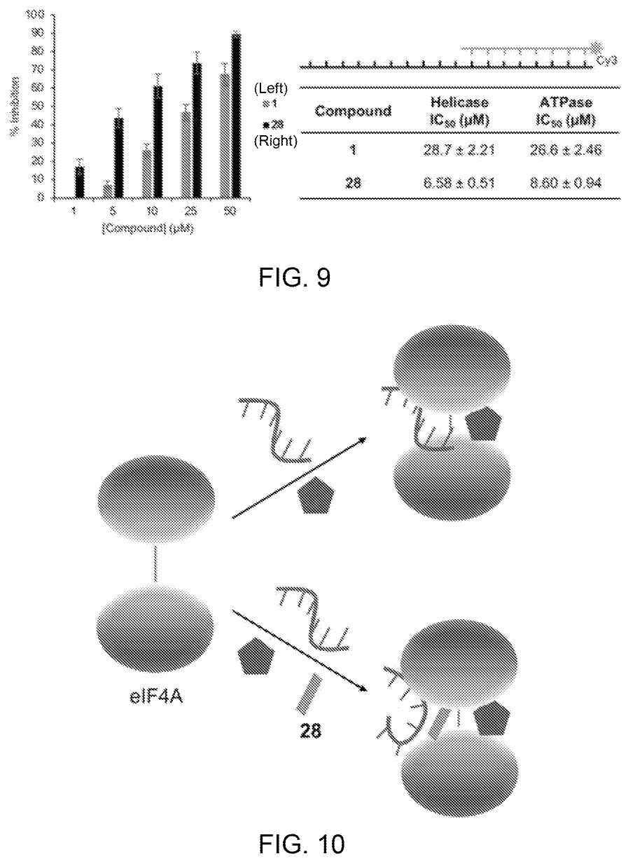

A functional duplex unwinding assay with a duplex nucleic acid substrate was utilized to test if compound 1 and compound 28 could inhibit eIF4A helicase activity. This unwinding assay features a Cy3-labeled RNA strand annealed to a DNA strand with a long overhang that eIF4A can bind (FIG. 9, top right). When ATP is added to protein and substrate, eIF4A unwinds the duplex, leaving an unlabeled DNA strand and a Cy3-labeled single stranded RNA. When visualized on a native gel, the larger intact fluorescent duplex substrate will run higher on the gel than the smaller unwound single-stranded fluorescent RNA. The ratio of these two Cy3 signals can be used to determine the helicase activity of eIF4A in each condition.

Dose responses of compound 1 and compound 28 were conducted using this assay. With no eIF4A or ATP, no unwinding takes place, and the Cy3 signal is present in the double-stranded state. Without compound, unwinding proceeds and the signal is present in the single-stranded state. Upon addition of increasing amounts of each compound, unwinding activity decreases, as shown by an increase in double-stranded signal and decrease in single-stranded signal. The intensity of each band in each gel lane was quantified by densitometry to obtain IC₅₀ values for compound 1 and compound 28 in this assay. Compound 28 was about four-fold more potent than compound 1, which was consistent with the other assays performed comparing these two compounds (FIG. 9). In addition, the IC₅₀ values for compound 1 and compound 28 in the helicase assay correlated very closely with both IC₅₀ values in the ATPase assay. This suggests that compound 1 and compound 28 inhibit eIF4A duplex unwinding as a direct result of inhibiting the ATPase activity of the protein. Altogether, compound 28 is an RNA-competitive, ATP-uncompetitive eIF4A inhibitor that binds a novel pocket in the RNA groove and inhibits eIF4A with a novel mechanism, in which it perturbs RNA binding, blocks the hydrolysis of ATP, and consequently, inhibits RNA helicase activity.

### Discussion

Compound 28 inhibits eIF4A through an RNA-competitive, ATP-uncompetitive mechanism that has not been observed for other eIF4A inhibitors. In the absence of an inhibitor, eIF4A exists in an open state until it cooperatively binds ATP and RNA. ATP hydrolysis then triggers RNA unwinding, a transition from the closed to the open state, and release of substrates. As an ATP-uncompetitive inhibitor of eIF4A, compound 28 prefers to bind eIF4A in the ATP-bound state. This cooperative nature of ATP and compound 28 binding matches the cooperative nature that ATP and RNA have for eIF4A binding. Once ATP and compound 28 are bound, RNA binds eIF4A in a manner that is either incomplete, or in a conformation that is inadequate for enzymatic activity of eIF4A (FIG. 10). With no ATP hydrolysis, eIF4A is unable to unwind RNA, and the RNA can more easily exit without being unwound.

The FP assay with the RNA fluorophore does not indicate that compound 28 enhances the interaction between eIF4A and RNA, but the potency of compound 28 in this assay is markedly lower than the other assays (FIG. 8C, middle). At high concentrations, compound 28 completely competes off. The reason for this is unknown, although it is possible that compound 28 may be able to occupy additional less-favorable binding sites within the RNA groove at high concentrations of inhibitor. At lower concentrations of compound 28, doses that inhibit ATPase activity and unwinding activity, RNA is still bound to eIF4A. These collective data strongly argue that eIF4A forms at least a pseudo-closed ATP-bound state with compound 28 and RNA engaging the RNA groove, and that RNA does not have to be completely competed off eIF4A for 28 to inhibit unwinding activity. The presence of compound 28 may force RNA to bind eIF4A incorrectly, with the entire RNA groove not occupied, placing the protein in a position incapable of ATP hydrolysis and RNA unwinding.

This mechanism of inhibition is unique among eIF4A inhibitors. Rocaglates inhibit eIF4A by increasing the affinity of eIF4A for RNA and clamping eIF4A shut on RNA. PatA acts similarly, also stabilizing the eIF4A-RNA interaction. In addition, PatA stimulates eIF4A ATPase and helicase activities and inhibits the association of eIF4A and eIF4G, sequestering eIF4A from the eIF4F complex. The mechanism of elatol is not explicitly known, but its interaction with K82 of eIF4A suggests it may be ATP-competitive. Hippuristanol is the only other RNA-competitive inhibitor of eIF4A, but unlike compound 28, it locks eIF4A closed and inhibits eIF4A unwinding activity by completely preventing RNA binding. Conversely, the reduced potency of compound 28 in the RNA FP assay, as discussed above, suggests that compound 28 inhibits eIF4A unwinding while incompletely inhibiting RNA binding.

Compound 28 also occupies a novel binding pocket within the eIF4A RNA-binding groove. Rocaglates bind a small cavity that is encompassed by the N-terminal domain of eIF4A and polypurine RNA. Desmethyl pateamine A (DMPatA), a PatA analog, occupies largely the same binding site as rocaglates, forming major interactions with RNA and the N-terminal domain of eIF4A. However, unlike rocaglates, DMPatA also extends across the RNA to form minor interactions with the C-terminal domain of eIF4A, and is able to clamp onto both polypurine and polypyrimidine RNA. Elatol binds eIF4A in a 2:1 stoichiometric fashion, with both elatol molecules predicted to reside near the eIF4A ATP pocket. Hippuristanol, although it shares an RNA-competitive mechanism with compound 28, binds exclusively to the C-terminal domain of eIF4A. The uncovering of a novel binding pocket provides additional molecular space that can be probed to produce additional novel eIF4A inhibitors.

An eIF4A inhibitor that is uncompetitive with respect to ATP has potential advantages in cells or in vivo. This uncompetitive mechanism means that compound 28 prefers to bind eIF4A when the protein is in the ATP-bound state, which is advantageous, as binding of ATP to eIF4A facilitates a closed state that forms a pocket or groove for either compound 28 or both compound 28 and RNA to bind. Intracellular ATP concentrations are typically anywhere from 1 to 10 mM, which poses a problem with ATP-competitive inhibitors in that it is difficult to successfully compete with such a high concentration of ATP for binding to the protein. However, for an ATP-uncompetitive inhibitor such as compound 28, the high concentration of ATP in the cell is a benefit for the molecule. eIF4A has a high likelihood of being ATP-bound in the cell, and compound 28 prefers binding eIF4A when ATP is present on the protein.

The EC₅₀ of compound 28 in BJAB Burkitt lymphoma cells is lower than the IC₅₀ values for the inhibitor in biochemical assays. It is possible that BJAB cells are extremely sensitive to eIF4A inhibition, and only a minor amount of eIF4A inhibition may be required to decrease cell viability. The BJAB cell viability tracking with biochemical inhibition of eIF4A for multiple analogs in the series, the ability of compound 28 to preferentially inhibit cap-dependent translation in cellular extracts, and the ability of compound 28 to engage eIF4A in cells provides strong evidence against major off-target effects leading to inhibition of BJAB cellular viability by compound 28. However, further work may be required to develop a deeper understanding of the intricacies and consequences of this mode of eIF4A inhibition. Compound 28 is a unique eIF4A inhibitor that operates through a novel mechanism and occupies a previously unreported eIF4A binding pocket. This molecule may give insight into the mechanism and interplay of eIF4A and its substrates, trigger biochemical probing into a new binding site that has potential pharmacological relevance, and uncover downstream cellular translational effects resulting from this novel mechanism of eIF4A inhibition.

### Experimental

Cloning and recombinant protein expression and purification. eIF4A was cloned into the pSpeedET expression vector (N-terminal 6×His tag and TEV protease site) using ligation independent PIPE cloning. BL21-CodonPlus cells were transformed with the desired plasmid and were used to inoculate 2×YT media containing 50 μg/ml kanamycin. This culture was grown to an OD₆₀₀ of 0.8 at 37° C. before being transferred to a 16° C. incubator. After 1 hour at 16° C., arabinose was added to a final concentration of 2 mg/mL and the cells were grown overnight. The next morning, cells were harvested by centrifugation and then resuspended in lysis buffer (50 mM HEPES pH 7.4, 150 mM KCl, 1 mM MgCl2, 10% glycerol, and 2 mM BME, 1× Protease Inhibitor Tablet [Pierce]). This slurry was then passed through a microfluidizer (LM10, Microfluidics Corporation) at 12,000 PSI three times and clarified by centrifugation. The supernatant was then incubated with cobalt talon resin (GoldBio) for 1 hr before being applied to a gravity column. This resin was then washed with 10 CV of lysis buffer and 10 CV of lysis buffer containing 5 mM imidazole. The desired protein was then eluted in lysis buffer containing 100 mM imidazole. His₆-TEV protease was then added to the His-tagged eIF4A and it was dialyzed against 20 mM Tris (pH 7.5), 10% glycerol, 0.1 mM EDTA, 2 mM DTT for 16 h at 4° C. to remove the His-tag. Protein was then incubated with Ni-NTA Agarose Resin (Qiagen) that had been washed with 10 CV dialysis buffer to recapture His₆-TEV and any uncut eIF4A and resin was removed by gravity filtration. eIF4A was aliquoted, flash frozen using liquid nitrogen, and stored at −80° C.

ATPase assay. Recombinant WT or mutant eIF4A was added to a clear bottom 384 well plate (Greiner) in ATPase buffer (20 mM Tris pH 7.4, 80 mM KCl, 2.5 mM MgCl₂, 1 mM DTT, 1% glycerol). Compounds were added and the plate was incubated at 37° C. for 20 min. ATP/RNA solution was added to afford a 20 μL per well reaction volume with 750 nM eIF4A, 250 μM ATP, and 0.25 mg/mL Yeast RNA (Sigma) for the initial screen and dose responses, and varying concentrations of ATP and RNA for the kinetic assays. The plate was incubated for 4 h at 37° C. for the initial screen and dose responses, and at seven time points (1 hour-7 hours) for kinetic assays. 0.04% v/v (final concentration) Tween-20 was added to a Malachite Green solution, and the Malachite Green (40 μL per well) was added. The solution was incubated for 5 minutes at room temperature and Absorbance at 660 nm was read on a SpectraMax iD5 plate reader (Molecular Devices).

Molecular docking. AutoDockTools version 1.5.6 (Scripps) was used for the docking simulations. MM2 energy-minimized molecules (compound 1, compound 28) were prepared in Chem3D version 16.0 (PerkinElmer). Lamarckian genetic algorithm was selected for ligand conformational searching, and rigid docking was performed on eIF4A (PDB: 5ZC9) first with RNA, rocaglate, and AMPPNP removed, and then with only RNA and rocaglate removed. Each molecule was docked 20 times, and the output of each docking simulation was the result of 2,500,000 evaluations. The lowest energy outputs for each interaction are displayed.

MANT-ATP fluorescence polarization assay. Recombinant eIF4A (15 μM) was added to a low-volume black 384 well plate (Greiner). Yeast RNA (0.25 mg/mL) was added along with compound (200 μM) or ADP (1 mM) and the plate was incubated for 20 min. MANT-ATP was added (100 nM) and the plate was incubated for 30 min before fluorescence polarization was read at 355 nm:448 nm (Ex:Em) on a SpectraMax iD5 plate reader (Molecular Devices).

FAM-RNA fluorescence polarization assay. Oligos were purchased from Integrated DNA Technologies using RNase free preparations. Recombinant eIF4A (2 μM) was incubated with 25 nM FAM-labelled RNA for 30 min in FP Buffer (15 mM HEPES-NaOH (pH 8), 100 mM NaCl, 1 mM MgCl₂, 15% glycerol, 2 mM DTT, 2 mM AMPPNP) at 22° C. in low-volume black 384 well plates (Greiner). eIF4A was incubated with compound for 20 min prior to the addition of FAM-RNA. Plate was incubated for another 30 min before fluorescence polarization was read at 485:535 nm (Ex:Em) on a SpectraMax iD5 plate reader (Molecular Devices. IC₅₀ values were calculated using GraphPad Prism v6.0.

Thermal Shift Assay (TSA)/Differential Scanning Fluorimetry (DSF). TSA/DSF experiments were performed on a Lightcycler 480 II (Roche Molecular Systems). eIF4A (5 μM) in buffer (20 mM Tris (pH 7.5), 10% glycerol, 0.1 mM EDTA, 2 mM DTT, 2 mM AMPPNP, 4% DMSO) was added to 96-well PCR plates (USA Scientific, Inc.) with or without compound. After a 5 min incubation, SYPRO orange dye was added to a final concentration of 5×. Samples were melted over a gradient from 20 to 85° C. with 20 acquisitions per ° C. at a ramp rate of 0.03° C./s.

Cell viability assay. BJAB lymphoma cells were plated in quadruplicate at 5,000 cells/well in serial dilutions of drug ranging two logs with the top concentration of compounds tested at 10 uM. Viability was measured after 72 h using Cell Titer Glo (Promega G7573) following the manufacturer's protocol. Luminescence was detected on the BioTek HT Synergy plate reader and values were normalized to vehicle-treated (DMSO) wells. Silvestrol was included as a positive control (not shown).

Cellular Thermal Shift Assay (CETSA). A549 cells were seeded into wells in 6-well plates and on the following day were exposed to 28 or silvestrol (100 nM) for 90 min. Cells were harvested, resuspended in 50 μL DMEM, and dispensed into thin-walled PCR tubes. Cells were subjected to 3 min of heat at 55° C. in a thermocycler (miniPCR). After heating, cells were left to cool for 3 min and then snap-frozen on dry ice. Cells were subjected to 3 rapid freeze thaws with 15 seconds of vortexing after each thaw. After the first thaw, 1 μL of a dissolved Pierce protease inhibitor tablet (ThermoFisher—A32963) was added to the cell lysate. Cell lysates were centrifuged at 20,000×g for 20 min. Samples were prepared for gel-electrophoresis and immunoblot analysis by transferring 40 μL of supernatant to 40 μL of 2× Laemmli (62.5 mM Tris-CI (pH 6.9), 3% SDS, 20% glycerol, 10% β-mercaptoethanol, and 0.1% bromophenol blue), sonicating for 30 seconds using a Fisher Scientific Sonic Dismembrator 100 (ThermoFisher), and boiling for 10 minutes. Cell lysates were then resolved by SDS-PAGE and subjected to immunoblot analysis using primary antibodies against eIF4A (Cell Signaling; 2490S) and GAPDH (Santa Cruz; sc-32233), and HRP-conjugated goat anti-rabbit (A0545) and goat anti-mouse (A9044) secondary antibodies (Sigma). All immunoblot images were taken using the Azure 600 imaging system (Azure Biosystems) and analyzed using ImageJ 1.51s (NIH). Gel band densitometry analysis was performed using ImageJ 1.53k (NIH).

### In Vitro Transcription and Translation

The dual-luciferase reporter plasmid pSP/(CAG)₃₃/FF/HCV/Ren-pA₅₁ was linearized with BamHI and subsequently in vitro transcribed using the Promega mMessage mMachine SP6 transcription kit, according to manufacturer's specifications. RNA was precipitated with LiCI and resuspended in H₂O for use in in vitro translation experiments. Rabbit reticulocyte lysate (RRL) (nuclease-treated, Promega) was used to assess inhibitory effects of 28 on translation of the dual-reporter. Briefly, a reaction consisting of 50% RRL, 30 ng/μL reporter RNA, 10 μM Amino Acid Mixture Minus Methionine, 10 μM Amino Acid Mixture Minus Leucine, 100 μM KCl, 1 U/μL RNAseOut (Thermo), and drug (final DMSO concentration 1%) were incubated at 30° C. for 90 minutes. The reaction was quenched with 50 μL 1× Passive Lysis Buffer (Promega), 10 μL of the mix was transferred to a 96-well white plate (Cellstar), and the Dual-Reporter Luciferase Assay System (Promega) was used to generate luminescence according to the manufacturer's instructions. Luminescence was detected using a plate reader (BioTek HT Synergy).

Helicase assay. Oligos were purchased from Integrated DNA Technologies using RNase free preparations. Unwinding reactions are performed in 50 ul volumes which contained 10 nM RNA/DNA duplex, 2.5 μM eIF4A, 2 mM ATP, 20 mM Tris (pH 7.5), 50 mM KCl, 1 mM DTT, 2 mM MgCl₂, and 0.1 mg/ml BSA. The sequence of the short RNA strand was 5′-GUGCUUUACGGUGCU-3′ and the 5′ end was amine modified for labeling with Cy3. The sequence of the DNA strand was: 5′-GGGAGAAAAACAAAACAAAACAAAACTAGCACCGTAAAGCACGC-3′. Reactions were incubated at 37° C. for 1 h and terminated by the addition of 12.5 μl of 50% glycerol, 2% SDS, 20 mM EDTA. For samples with no eIF4A, Cy3-RNA and DNA were mixed at 1 μM, placed in a 90° C. heat block, and the block was allowed to cool to room temp over 90 min. The samples were then placed on ice for 10 min, diluted to 50 nM and termination buffer was added. All products were analyzed by electrophoresis on 20% native polyacrylamide (37.5:1 acrylamide:bisacrylamide) gels. Gels were run at 4° C. for 2 hours and Cy3 signal was detected on an Azure 600 Fluorescent Imaging System (Azure Biosystems) at 524 nm:572 nm (Ex:Em). Cy3 signal was quantified by gel band densitometry performed using ImageJ32 (NIH). IC₅₀ values were calculated using GraphPad Prism v6.0.

### Chemistry

All chemicals that were commercially available were used without further purification. Detailed synthesis procedures can be found below for reactions described in Schemes 1-7. All reaction intermediates were purified by flash column chromatography. ¹H and ¹¹C NMR spectra were recorded on a Bruker AVIII-400 or Bruker DRX-500 NMR spectrometer. Coupling constants (J) are reported in hertz (Hz). Chemical shifts (6) are reported in parts per million referenced with respect to residual solvent (DMSO-d₆) 2.50 ppm and (Chloroform-d) 7.24 ppm or from internal standard tetramethylsilane (TMS) 0.00 ppm. The following abbreviations were used in reporting spectra: s, singlet; d, doublet; t, triplet; q, quartet; m, multiplet; dd, doublet of doublets; td, triplet of doublets; ddd, doublet of doublet of doublets; p, pentet; h, hextet. HPLC-grade solvents were used for all reactions. NMR data were processed using MestReNova 14.2.0 (Mestrelab). Flash column chromatography was performed using silica gel (230-400 mesh, Agela). High-resolution mass spectrometry (HRMS) was performed using a Q Exactive Plus Orbitrap MS system (Thermo Fisher). The mass spectrometer was operated in electrospray positive ionization mode (ESI+). Acquisition was a scan from m/z 100 to 1000 with an accumulation time of 2 minutes. The purity was assessed using an Agilent 1200 Series HPLC with an Alltech Econosphere Silica 5 u column (cat no. 70006), 250×4.6 mm, at a flow rate of 0.75 mL/min; λ=430 nm; mobile phase: 95% dichloromethane, 5% methanol. All compounds were characterized by HRMS, proton NMR, and carbon NMR unless low yield prevented it and were confirmed >95.0% pure by HPLC traces.

### Synthesis PROCEDURES

### 1-(5-bromofuran-2-yl)ethan-1-one (1i)

To a round bottom flask was added 2-acetylfuran (2.75 g, 25 mmol). A solution of N-bromosuccinimide (4.89 g, 27.5 mmol) in DMF (5 mL) was added dropwise over a period of 15 minutes and the reaction stirred at room temperature overnight. Ethyl acetate was added, and the reaction was washed with brine, dried (MgSO₄), filtered, and concentrated in vacuo. The product was purified by silica gel column chromatography (95:5 Hexanes:EtOAc) to afford the final product (white powder, 2.5 g, 13.2 mmol, 53% yield). ¹H NMR (400 MHz, Chloroform-d) δ 7.14 (d, J=3.6 Hz, 1H), 6.51 (d, J=3.6 Hz, 1H), 2.49 (s, 3H). ¹³C NMR (101 MHz, Chloroform-d) δ 185.48, 154.55, 128.17, 118.90, 114.38, 25.75. HRMS (ESI) calcd for [M+H]⁺, 188.9473; found 188.9506.

General procedure for the production of advanced acetylated ring intermediates (1ii, 31i, 33i-45i, 48i-54i).

To a stirred solution of a boronic acid-substituted ring (3 mmol), Cs₂CO₃ (978 mg, 3 mmol), acetylated, bromo-substituted ring (1.5 mmol), and 9:1 toluene:water was added tetrakis(triphenylphosphine)palladium(0) (87 mg, 5 mol %,) and the reaction stirred overnight at 100° C. The mixture was filtered through celite and rinsed with ethyl acetate, and the filtrate was concentrated in vacuo. The product was purified by silica gel column chromatography to afford the final product.

### 1-(5-(4-butylphenyl)furan-2-yl)ethan-1-one (1ii)

¹H NMR (400 MHz, Chloroform-d) δ 7.73 (d, 2H), 7.31-7.23 (m, 3H), 6.75 (d, J=3.7 Hz, 1H), 2.67 (t, 2H), 1.65 (p, 2H), 1.40 (h, 2H), 0.97 (t, 3H). ¹³C NMR (101 MHz, Chloroform-d) δ 186.34, 158.08, 151.67, 144.50, 128.95, 126.86, 124.97, 119.60, 106.82, 35.53, 33.42, 25.93, 22.32, 13.93. HRMS (ESI) calcd for [M+H]⁺, 243.1307; found 243.13798.

### 1-(5-(4-butylphenyl)thiophen-2-yl)ethan-1-one (31i)

¹H NMR (500 MHz, Chloroform-d) δ 7.60 (d, J=3.9 Hz, 1H), 7.54-7.50 (m, 2H), 7.24 (d, J=4.0 Hz, 1H), 7.20-7.16 (m, 2H), 2.59 (t, J=7.7 Hz, 2H), 2.51 (s, 3H), 1.61-1.53 (m, 2H), 1.33 (h, 2H), 0.89 (t, J=7.4 Hz, 3H). HRMS (ESI) calcd for C₁₆H₁₉OS [M+H]⁺, 259.1078; found 259.1093.

### 1-(4′-butyl-[1,1′-biphenyl]-3-yl)ethan-1-one (33i)

¹H NMR (400 MHz, Chloroform-d) δ 8.20 (t, J=1.8 Hz, 1H), 7.97-7.91 (m, 1H), 7.81 (ddd, J=7.7, 1.9, 1.1 Hz, 1H), 7.60-7.51 (m, 3H), 7.34-7.29 (m, 2H), 2.75-2.66 (m, 5H), 1.68 (h, 2H), 1.43 (h, J=7.3 Hz, 2H), 0.99 (t, J=7.3 Hz, 3H). HRMS (ESI) calcd for C₁₈H₂₁O [M+H]⁺, 253.1514; found 253.1563.

### 1-(6-(4-butylphenyl)pyridin-2-yl)ethan-1-one (34i)

¹H NMR (500 MHz, Chloroform-d) δ 7.95-7.90 (m, 2H), 7.85 (dd, J=7.3, 1.4 Hz, 1H), 7.82-7.73 (m, 2H), 7.26-7.20 (m, 2H), 2.74 (s, 3H), 2.60 (t, J=7.7 Hz, 2H), 1.61-1.51 (m, 2H), 1.31 (h, J=7.3 Hz, 2H), 0.87 (t, J=7.3 Hz, 3H). HRMS (ESI) calcd for C₁₇H₂₀NO [M+H]⁺, 254.1467; found 254.1501.

### 1-(5-(4-butylphenyl)pyridin-3-yl)ethan-1-one (35i)

¹H NMR (400 MHz, Chloroform-d) δ 9.13 (d, J=2.1 Hz, 1H), 9.03 (d, J=2.3 Hz, 1H), 8.46 (t, J=2.2 Hz, 1H), 7.61-7.52 (m, 2H), 7.39-7.31 (m, 2H), 2.77-2.67 (m, 5H), 1.73-1.62 (m, 2H), 1.42 (h, J=7.3 Hz, 2H), 0.98 (t, J=7.3 Hz, 3H). HRMS (ESI) calcd for C₁₇H₂₀NO [M+H]⁺, 254.1467; found 254.1499.

### 1-(4′-butyl-[1,1′-biphenyl]-4-yl)ethan-1-one (36i)

¹H NMR (500 MHz, Chloroform-d) δ 7.99-7.94 (m, 2H), 7.66-7.60 (m, 2H), 7.52-7.47 (m, 2H), 7.26-7.22 (m, 2H), 2.65-2.59 (m, 2H), 2.58 (s, 3H), 1.65-1.55 (m, 2H), 1.34 (h, J=7.4 Hz, 2H), 0.90 (t, J=7.4 Hz, 3H). HRMS (ESI) calcd for C₁₈H₂₁O [M+H]⁺, 253.1514; found 253.1542.

### 1-(6-(4-butylphenyl)pyridin-3-yl)ethan-1-one (37i)

¹H NMR (500 MHz, Chloroform-d) δ 9.13 (dd, J=2.4, 0.9 Hz, 1H), 8.23 (dd, J=8.4, 2.3 Hz, 1H), 7.94-7.88 (m, 2H), 7.75 (dd, J=8.4, 0.8 Hz, 1H), 7.26-7.20 (m, 2H), 2.61-2.54 (m, 5H), 1.59-1.49 (m, 2H), 1.28 (h, J=7.4 Hz, 2H), 0.84 (t, J=7.4 Hz, 3H). HRMS (ESI) calcd for C₁₇H₂₀NO [M+H]⁺, 254.1467; found 254.1485.

### 1-(5-phenylfuran-2-yl)ethan-1-one (38i)

¹H NMR (500 MHz, Chloroform-d) δ 7.79-7.73 (m, 2H), 7.43-7.38 (m, 2H), 7.37-7.31 (m, 1H), 7.24 (d, J=3.7 Hz, 1H), 6.75 (d, J=3.7 Hz, 1H), 2.51 (s, 3H). HRMS (ESI) calcd for C₁₂H₁₁O₂[M+H]⁺, 187.0681; found 187.0712.

### 1-(5-(4-propylphenyl)furan-2-yl)ethan-1-one (39i)

¹H NMR (400 MHz, DMSO-d₆) δ 7.80-7.73 (m, 2H), 7.55 (d, J=3.7 Hz, 1H), 7.36-7.27 (m, 2H), 7.15 (d, J=3.7 Hz, 1H), 2.64 (t, J=7.7 Hz, 2H), 2.46 (s, 3H), 1.62 (h, 2H), 0.91 (t, J=7.3 Hz, 3H). HRMS (ESI) calcd for C₁₅H₁₇O₂[M+H]⁺, 229.1150; found 229.1188.

### 1-(5-(4-pentylphenyl)furan-2-yl)ethan-1-one (40i)

¹H NMR (400 MHz, DMSO-d₆) δ 7.81-7.73 (m, 2H), 7.55 (d, J=3.7 Hz, 1H), 7.36-7.28 (m, 2H), 7.14 (d, J=3.7 Hz, 1H), 2.64 (t, J=7.7 Hz, 2H), 2.46 (s, 3H), 1.62 (h, 2H), 1.38-1.23 (m, 4H), 0.91 (t, J=7.3 Hz, 3H). HRMS (ESI) calcd for C₁₇H₂₀O₂[M+H]⁺, 257.1463; found 257.1490.

### 1-(5-(4-propoxyphenyl)furan-2-yl)ethan-1-one (41i)

¹H NMR (400 MHz, Chloroform-d) δ 7.77-7.70 (m, 2H), 7.30-7.25 (m, 1H), 7.00-6.93 (m, 2H), 6.66 (d, J=3.7 Hz, 1H), 3.99 (t, J=6.6 Hz, 2H), 2.54 (s, 3H), 1.87 (h, 2H), 1.09 (t, J=7.4 Hz, 3H). HRMS (ESI) calcd for C₁₅H₁₇O₃[M+H]⁺, 245.1099; found 257.1126.

### 1-(5-(3-propoxyphenyl)furan-2-yl)ethan-1-one (42i)

¹H NMR (400 MHz, Chloroform-d) δ 7.43-7.32 (m, 3H), 7.28 (d, J=3.7 Hz, 1H), 6.94 (ddd, J=7.6, 2.6, 1.6 Hz, 1H), 6.79 (d, J=3.7 Hz, 1H), 4.01 (t, J=6.5 Hz, 2H), 2.54 (s, 3H), 1.86 (h, J=7.1 Hz, 2H), 1.10 (t, J=7.4 Hz, 3H). HRMS (ESI) calcd for C₁₅H₁₇O₃[M+H]⁺, 245.1099; found 257.1129.

### 1-(5-(2-propoxyphenyl)furan-2-yl)ethan-1-one (43i)

¹H NMR (400 MHz, Chloroform-d) δ 8.03 (dd, J=7.8, 1.7 Hz, 1H), 7.34 (ddd, J=8.4, 7.3, 1.8 Hz, 1H), 7.31-7.27 (m, 1H), 7.13 (d, J=3.7 Hz, 1H), 7.06 (td, J=7.6, 1.1 Hz, 1H), 6.99 (dd, J=8.3, 1.1 Hz, 1H), 4.10 (t, J=6.5 Hz, 2H), 2.56 (s, 3H), 1.97 (h, 2H), 1.14 (t, J=7.4 Hz, 3H). HRMS (ESI) calcd for C₁₅H₁₇O₃[M+H]⁺, 245.1099; found 257.1137.

### tert-butyl (4-(5-acetylfuran-2-yl)phenyl)carbamate (44i)

¹H NMR (500 MHz, Chloroform-d) δ 7.73-7.68 (m, 1H), 7.44 (d, J=8.4 Hz, 2H), 7.24 (d, J=3.6 Hz, 1H), 6.68 (d, J=3.6 Hz, 2H), 2.51 (s, 3H), 1.53 (s, 9H). HRMS (ESI) calcd for C₁₇H₂₀NO₄ [M+H]⁺, 302.1314; found 302.1368.

### tert-butyl (3-(5-acetylfuran-2-yl)phenyl)carbamate (45i)

¹H NMR (500 MHz, Chloroform-d) δ 7.80 (t, J=2.0 Hz, 1H), 7.45 (dt, J=7.6, 1.5 Hz, 1H), 7.39 (d, J=8.2 Hz, 1H), 7.33 (t, J=7.9 Hz, 1H), 7.24 (d, J=3.7 Hz, 1H), 6.78 (d, J=3.7 Hz, 1H), 2.52 (s, 3H), 1.53 (s, 9H). HRMS (ESI) calcd for C₁₇H₂₀NO₄ [M+H]⁺, 302.1314; found 302.1362.

### 1-(5-(benzo[d][1,3]dioxol-5-yl)furan-2-yl)ethan-1-one (48i)

¹H NMR (400 MHz, DMSO-d₆) δ 7.52 (d, J=3.7 Hz, 1H), 7.43-7.42 (m, 1H), 7.39 (dd, J=8.1, 1.8 Hz, 1H), 7.09 (d, J=3.7 Hz, 1H), 7.05 (dd, J=8.1, 0.4 Hz, 1H), 6.11 (s, 2H), 2.45 (s, 3H). HRMS (ESI) calcd for C₁₃H₁₁O₄[M+H]⁺, 231.0579; found 231.0602.

### 1-(5-(naphthalen-1-yl)furan-2-yl)ethan-1-one (49i)

¹H NMR (500 MHz, Chloroform-d) δ 8.26 (dt, J=8.6, 1.0 Hz, 1H), 7.81-7.75 (m, 2H), 7.72-7.68 (m, 1H), 7.46-7.39 (m, 3H), 7.24 (d, J=3.6 Hz, 1H), 6.73 (d, J=3.6 Hz, 1H), 2.44 (s, 3H). HRMS (ESI) calcd for C₁₆H₁₃O₂[M+H]⁺, 237.0837; found 237.0854.

### 1-(5-([1,1′-biphenyl]-4-yl)furan-2-yl)ethan-1-one (50i)

¹H NMR (400 MHz, DMSO-d₆) δ 7.99-7.92 (m, 2H), 7.87-7.80 (m, 2H), 7.79-7.71 (m, 2H), 7.59 (d, J=3.7 Hz, 1H), 7.54-7.46 (m, 2H), 7.45-7.37 (m, 1H), 7.28 (d, J=3.6 Hz, 1H), 2.49 (s, 3H). HRMS (ESI) calcd for C₁₈H₁₅O₂[M+H]⁺, 263.0994; found 263.1015.

### 1-(5-([1,1′-biphenyl]-3-yl)furan-2-yl)ethan-1-one (51i)

¹H NMR (400 MHz, DMSO-d₆) δ 8.11 (td, J=1.8, 0.5 Hz, 1H), 7.86 (ddd, J=7.7, 1.8, 1.1 Hz, 1H), 7.79-7.73 (m, 2H), 7.72 (ddd, J=7.8, 1.8, 1.1 Hz, 1H), 7.64-7.56 (m, 2H), 7.55-7.49 (m, 2H), 7.46-7.39 (m, 1H), 7.36 (d, J=3.7 Hz, 1H), 2.49 (s, 3H). HRMS (ESI) calcd for C₁₈H₁₅O₂[M+H]⁺, 263.0994; found 263.1037.

### 1-(5-(4-phenoxyphenyl)furan-2-yl)ethan-1-one (52i)

¹H NMR (400 MHz, Chloroform-d) δ 7.81-7.75 (m, 2H), 7.44-7.37 (m, 2H), 7.30-7.27 (m, 1H), 7.19 (ddt, J=8.4, 7.6, 1.2 Hz, 1H), 7.11-7.04 (m, 4H), 6.72 (d, J=3.7 Hz, 1H), 2.55 (s, 3H). HRMS (ESI) calcd for C₁₈H₁₅O₃[M+H]⁺, 279.0943; found 279.0980.

### 1-(5-(3-phenoxyphenyl)furan-2-yl)ethan-1-one (53i)

¹H NMR (400 MHz, Chloroform-d) δ 7.59-7.53 (m, 1H), 7.47 (dd, J=2.5, 1.6 Hz, 1H), 7.43-7.35 (m, 3H), 7.27 (d, J=3.7 Hz, 1H), 7.16 (ddt, J=7.5, 5.9, 1.1 Hz, 1H), 7.09-7.04 (m, 2H), 7.01 (ddd, J=8.1, 2.5, 1.0 Hz, 1H), 6.77 (d, J=3.7 Hz, 1H), 2.54 (s, 3H). HRMS (ESI) calcd for C₁₈H₁₅O₃[M+H]⁺, 279.0943; found 279.0971.

### 1-(5-(2-phenoxyphenyl)furan-2-yl)ethan-1-one (54i)

¹H NMR (400 MHz, Chloroform-d) δ 8.09 (dd, J=7.8, 1.8 Hz, 1H), 7.40-7.33 (m, 2H), 7.33-7.26 (m, 1H), 7.25-7.19 (m, 2H), 7.14 (tt, J=7.4, 1.1 Hz, 1H), 7.07-7.00 (m, 3H), 6.95 (dd, J=8.2, 1.3 Hz, 1H), 2.52 (s, 3H). HRMS (ESI) calcd for C₁₈H₁₅O₃[M+H]⁺, 279.0943; found 279.0984.

### 1-(5-bromo-1H-pyrrol-2-yl)ethan-1-one (321)

Oxone (7.15 g, 11.6 mmol) and NaBr (2.99 g, 29.1 mmol) were added to a solution of 2-acetylpyrrole (2.54 g, 23.3 mmol) in a mixture of methanol (50 mL) and water (50 mL). The reaction stirred for 8 hours at room temperature. The resulting mixture was filtered, and the filtrate was extracted with dichloromethane. The organic layer was dried (Na₂SO₄) and filtered. The solvent was removed in vacuo and the residue was purified by silica gel chromatography (90:10 Hexanes:EtOAc) to afford the final product (white solid, 1.68 g, 8.91 mmol, 38% yield). ¹H NMR (400 MHz, Chloroform-d) δ 7.04-6.99 (m, 1H), 6.93-6.87 (m, 1H), 2.41 (s, 3H). HRMS (ESI) calcd for C₆H₇BrNO [M+H]⁺, 187.9633; found, 187.9657.

### tert-butyl 2-acetyl-5-bromo-1H-pyrrole-1-carboxylate (3211)

1-(5-bromo-1H-pyrrol-2-yl)ethan-1-one (321, 2.84 g, 15 mmol) was dissolved in triethylamine (2.1 mL) and 4-dimethylaminopyridine (36.5 mg, 2 mol %) was added. Di-tert-butyl dicarbonate (3.27 g, 15 mmol) was dissolved in 15 mL THE and added to the reaction. This reaction stirred overnight at room temperature. The resulting mixture was diluted with 15 mL of saturated sodium bicarbonate, extracted with ether, dried (Na₂SO₄), and filtered. The solvent was removed in vacuo and the residue was purified by silica gel chromatography (96:4 Hexanes:EtOAc) to afford the final product (white solid, 0.96 g, 0.33 mmol, 22% yield). ¹H NMR (400 MHz, Chloroform-d) δ 7.32 (d, J=1.8 Hz, 1H), 6.81 (d, J=1.8 Hz, 1H), 2.46 (s, 3H), 1.60 (s, 9H). HRMS (ESI) calcd for C₁₁H₁₅BrNO₃ [M+H]⁺, 288.0157; found 288.0210.

### tert-butyl 2-acetyl-5-(4-butylphenyl)-1H-pyrrole-1-carboxylate (32111)

To a stirred solution of 4-butylphenylboronic acid (356 mg, 2 mmol), CsₛCO₃ (652 mg, 2 mmol), tert-butyl 2-acetyl-5-bromo-1H-pyrrole-1-carboxylate (3211, 285 mg, 1 mmol), 8 mL dioxane, and 2 mL water was added tetrakis(triphenylphosphine)palladium(0) (116 mg, 10 mol %,) and the reaction stirred overnight at 100° C. The mixture was filtered through celite and rinsed with ethyl acetate, and the filtrate was concentrated in vacuo. The product was purified by silica gel column chromatography (95:5 Hexanes:EtOAc) to afford the final product (amber solid, 157 mg, 0.46 mmol, 46% yield). ¹H NMR (400 MHz, Chloroform-d) δ 7.57 (d, J=1.8 Hz, 1H), 7.46-7.41 (m, 2H), 7.24-7.19 (m, 2H), 6.79-6.74 (m, 1H), 2.69-2.61 (m, 2H), 2.53 (s, 3H), 1.73-1.64 (m, 2H), 1.63 (s, 9H), 1.39 (h, J=7.3 Hz, 2H), 0.98 (t, J=7.3 Hz, 3H). HRMS (ESI) calcd for C₂₁H₂₇NO₃ [M+H]⁺, 342.1991; found, 342.2017.

### 1-(5-(4-butylphenyl)-1H-pyrrol-2-yl)ethan-1-one (32iv)

tert-butyl 2-acetyl-5-(4-butylphenyl)-1H-pyrrole-1-carboxylate (32111, 100 mg, 0.3 mmol) was dissolved in a 3 mL solution of 50:50 TFA:dichloromethane. The reaction stirred at room temperature and was monitored by TLC (80:20 Hexanes:EtOAc). When the reactant was consumed, saturated sodium bicarbonate (10 mL) was added, the reaction was extracted with dichloromethane, washed with brine, dried (Na₂SO₄), filtered, and purified via silica gel chromatography (85:15 Hexanes:EtOAc) to afford the product (white solid, 65 mg, 0.27 mmol, 90% yield). ¹H NMR (400 MHz, CDCl₃) δ 7.49-7.43 (m, 2H), 7.30 (dd, J=3.0, 1.6 Hz, 1H), 7.24-7.20 (m, 2H), 7.17 (dd, J=2.6, 1.6 Hz, 1H), 2.64 (t, J=7.7 Hz, 2H), 2.51 (s, 3H), 1.71-1.60 (m, 2H), 1.41 (h, J=7.3 Hz, 2H), 0.97 (t, J=7.3 Hz, 3H). HRMS (ESI) calcd for C₁₆H₂₀NO [M+H]⁺, 242.1467; found, 242.1498.

### 5-aminoisatin (241)

5-nitroisatin (500 mg, 2.6 mmol) was dissolved in warm ethanol (10 mL), and reduced iron powder was added. As the reaction stirred under argon, concentrated HCl (2 mL) was added dropwise, and the reaction mixture stirred for 3 hours at 75° C. The mixture was treated with saturated sodium bicarbonate and brine, and extracted with ethyl acetate. The organic layer was dried (MgSO₄), filtered, and concentrated in vacuo. The crude product was purified by silica gel chromatography (97:3 dichloromethane:methanol) to afford the final product (dark red solid, 170 mg, 1 mmol, 40% yield). ¹H NMR (400 MHz, DMSO-d₆) δ 10.58 (s, NH), 6.83 (dd, J=2.5, 8.3 Hz, 1H), 6.72 (d, J=2.4 Hz, 1H), 6.64 (d, J=8.2 Hz, 1H), 5.08 (s, 2NH). ¹³C NMR (101 MHz, DMSO) δ 185.86, 159.84, 145.33, 141.33, 124.04, 118.57, 113.18, 109.56. HRMS (ESI) calcd for C₈H6N₂O₂[M+H]⁺, 163.0429; found, 163.05020.

### 5-acetamidoisatin (25i)

5-aminoisatin (40 mg, 0.25 mmol) was dissolved in pyridine (1.5 mL) and stirred at 0° C. under argon. Acetic anhydride (28 μL, 0.25 mmol) was added dropwise, and the reaction stirred for 3 hr. A saturated solution of sodium bicarbonate was added (1.5 mL), and the mixture was extracted with ethyl acetate, dried (MgSO₄) and concentrated in vacuo. The residue was purified by silica gel chromatography (50:50 Hexanes:EtOAc) to isolate the final product (orange solid, 11 mg, 0.054 mmol, 22% yield). ¹H NMR (400 MHz, Acetone-d₆) δ 9.93 (s, NH), 9.28 (s, NH), 7.95 (d, J=2.2 Hz, 1H), 7.78 (dd, J=2.3, 8.4 Hz, 1H), 6.98 (d, J=8.5 Hz, 1H), 2.08 (s, 3H). HRMS (ESI) calcd for C₁₀H8N₂O₃[M+H]⁺, 204.0535; found, 204.05629.

General Procedure for the Production of Carboxylic Acid eIF4A Inhibitor Analogs (1, 3-28, 30-45, 48-54).

To a stirred solution of an isatin (0.37 mmol) in ethanol (0.87 mL), was added a solution of KOH (62.3 mg) in water (0.13 mL). The reaction stirred for 20 minutes, an advanced acetylated ring intermediate (0.37 mmol) was added slowly, and the reaction stirred overnight at 90° C. Water (1 mL) was added, and glacial acetic acid was added in 20 μL increments to form a precipitate. The precipitate was filtered and washed with and dichloromethane and acetone to afford the product.

### 6-bromo-2-(5-(4-butylphenyl)furan-2-yl)quinoline-4-carboxylic acid (1)

¹H NMR (400 MHz, DMSO-d₆) δ 8.91 (d, J=2.2 Hz, 1H), 8.44 (s, 1H), 8.04 (d, J=9.0 Hz, 1H), 7.96 (dd, J=9.0, 2.3 Hz, 1H), 7.82 (d, J=8.2 Hz, 2H), 7.59 (d, J=3.6 Hz, 1H), 7.34 (d, J=8.2 Hz, 2H), 7.19 (d, J=3.6 Hz, 1H), 2.64 (t, J=7.7 Hz, 2H), 1.60 (p, 2H), 1.35 (h, J=7.3 Hz, 2H), 0.93 (t, J=7.3 Hz, 3H). ¹³C NMR (126 MHz, DMSO-d₆) δ 167.43, 156.17, 151.83, 148.89, 147.71, 143.50, 133.95, 131.88, 129.50, 129.32, 127.59, 125.00, 124.62, 121.23, 119.74, 114.83, 34.98, 33.29, 22.07, 14.09. HRMS (ESI) calcd for C₂₄H₂₀BrNO₃ [M+H]⁺, 450.0627; found, 450.0699.

### 2-(5-(4-butylphenyl)furan-2-yl)quinoline-4-carboxylic acid (3)

¹H NMR (500 MHz, DMSO-d₆) δ 8.62 (dd, J=1.4, 8.5 Hz, 1H), 8.38 (s, 1H), 8.11 (dt, J=1.2, 8.4 Hz, 1H), 7.88-7.80 (m, 3H), 7.68 (ddd, J=1.3, 6.8, 8.3 Hz, 1H), 7.56 (d, J=3.6 Hz, 1H), 7.35 (d, J=8.3 Hz, 2H), 7.19 (d, J=3.5 Hz, 1H), 2.65 (t, J=7.7 Hz, 2H), 1.60 (p, 2H), 1.35 (h, 2H), 0.93 (t, J=7.4 Hz, 3H). HRMS (ESI) calcd for C₂₄H₂₂NO₃ [M+H]⁺, 372.1521; found 372.15961.

### 2-(5-(4-butylphenyl)furan-2-yl)-5-chloroquinoline-4-carboxylic acid (4)

¹H NMR (400 MHz, DMSO-d₆) δ 8.12 (s, 1H), 8.07 (dd, J=1.4, 8.2 Hz, 1H), 7.89-7.83 (m, 2H), 7.83-7.72 (m, 2H), 7.61 (d, J=3.6 Hz, 1H), 7.33 (d, J=8.2 Hz, 2H), 7.19 (d, J=3.6 Hz, 1H), 2.64 (t, J=7.7 Hz, 2H), 1.60 (p, J=7.5 Hz, 2H), 1.35 (h, J=7.3 Hz, 2H), 0.93 (t, J=7.3 Hz, 3H). ¹³C NMR (101 MHz, DMSO-d₆) δ 169.89, 157.20, 151.63, 151.51, 149.60, 146.58, 145.67, 143.42, 131.02, 129.39, 127.57, 124.71, 124.58, 120.76, 120.20, 108.70, 100.07, 35.11, 33.44, 22.22, 14.25. HRMS (ESI) calcd for C₂₄H₂₁ClNO₃ [M+H]⁺, 406.1132; found 406.12039.

### 2-(5-(4-butylphenyl)furan-2-yl)-6-fluoroquinoline-4-carboxylic acid (5)

¹H NMR (400 MHz, DMSO-d₆) δ 8.50-8.40 (m, 2H), 8.18 (dd, J=5.8, 9.3 Hz, 1H), 7.85-7.73 (m, 3H), 7.55 (d, J=3.6 Hz, 1H), 7.34 (d, J=8.2 Hz, 2H), 7.18 (d, J=3.6 Hz, 1H), 2.64 (t, J=7.7 Hz, 2H), 1.60 (p, 2H), 1.35 (h, J=7.4 Hz, 2H), 0.93 (t, J=7.3 Hz, 3H). ¹³C NMR (126 MHz, DMSO-d₆) δ 167.53, 159.96, 156.22, 155.86, 151.90, 148.07, 146.37, 143.39, 135.63, 135.54, 132.66, 129.48, 127.66, 124.56, 119.70, 114.19, 108.71, 34.98, 33.30, 22.07, 14.09. HRMS (ESI) calcd for C₂₄H₂₁FNO₃ [M+H]⁺, 390.1427; found 390.15016.

### 2-(5-(4-butylphenyl)furan-2-yl)-6-chloroquinoline-4-carboxylic acid (6)

¹H NMR (400 MHz, DMSO-d₆) δ 8.75 (d, J=2.4 Hz, 1H), 8.45 (s, 1H), 8.11 (d, J=9.0 Hz, 1H), 7.85 (dd, J=9.0, 2.4 Hz, 1H), 7.82 (d, J=8.2 Hz, 2H), 7.58 (d, J=3.6 Hz, 1H), 7.34 (d, J=8.2 Hz, 2H), 7.19 (d, J=3.6 Hz, 1H), 2.64 (t, J=7.7 Hz, 2H), 1.60 (p, J=7.8, 6.5 Hz, 2H), 1.35 (h, J=7.3 Hz, 2H), 0.93 (t, J=7.3 Hz, 3H). ¹³C NMR (126 MHz, DMSO-d₆) δ 167.47, 158.56, 156.13, 151.84, 148.85, 148.82, 147.55, 147.22, 143.56, 136.86, 132.50, 132.30, 131.80, 131.39, 130.15, 129.51, 128.80, 127.60, 124.62, 108.82, 34.98, 33.30, 22.06, 14.09. HRMS (ESI) calcd for C₂₄H₂₁ClNO₃ [M+H]⁺, 406.1132; found 406.12030.

### 2-(5-(4-butylphenyl)furan-2-yl)-6-iodoquinoline-4-carboxylic acid (7)

¹H NMR (400 MHz, DMSO-d₆) δ 9.12 (d, J=2.0 Hz, 1H), 8.29 (s, 1H), 8.03 (dd, J=2.0, 8.8 Hz, 1H), 7.85-7.77 (m, 3H), 7.52 (d, J=3.6 Hz, 1H), 7.37-7.30 (m, 2H), 7.17 (d, J=3.6 Hz, 1H), 2.64 (t, J=7.7 Hz, 2H), 1.60 (p, J=6.4, 7.9 Hz, 2H), 1.35 (h, J=7.4 Hz, 2H), 0.93 (t, J=7.4 Hz, 3H). ¹³C NMR (126 MHz, DMSO-d₆) δ 169.98, 161.77, 159.26, 159.12, 157.73, 156.58, 155.91, 152.49, 150.82, 147.57, 137.77, 136.44, 135.21, 133.19, 129.56, 127.47, 126.30, 124.55, 102.95, 95.87, 35.05, 33.28, 22.12, 14.10. HRMS (ESI) calcd for C₂₄H₂₁INO₃ [M+H]⁺, 498.0488; found 498.05611.

### 2-(5-(4-butylphenyl)furan-2-yl)-7-fluoroquinoline-4-carboxylic acid (8)

¹H NMR (400 MHz, DMSO-d₆) δ 8.72 (dd, J=6.4, 9.4 Hz, 1H), 8.37 (s, 1H), 7.87-7.80 (m, 3H), 7.64-7.59 (m, 1H), 7.58 (d, J=3.6 Hz, 1H), 7.37-7.33 (m, 2H), 7.20 (d, J=3.6 Hz, 1H), 2.65 (t, J=7.7 Hz, 2H), 1.61 (p, 2H), 1.35 (h, J=7.4, 14.6 Hz, 2H), 0.93 (t, J=7.3 Hz, 3H). ¹³C NMR (126 MHz, DMSO-d₆) δ 170.45, 167.12, 162.98, 147.68, 142.28, 139.90, 133.70, 131.56, 129.53, 129.10, 126.09, 124.64, 120.97, 119.26, 117.32, 116.36, 114.52, 112.69, 111.73, 108.75, 33.35, 23.26, 22.11, 14.09. HRMS (ESI) calcd for C₂₄H₂₁FNO₃ [M+H]⁺, 390.1427; found 390.15007.

### 2-(5-(4-butylphenyl)furan-2-yl)-7-chloroquinoline-4-carboxylic acid (9)

¹H NMR (400 MHz, DMSO-d₆) δ 8.68 (d, J=9.1 Hz, 1H), 8.36 (s, 1H), 8.12 (d, J=2.2 Hz, 1H), 7.85-7.79 (m, 2H), 7.68 (dd, J=2.2, 9.1 Hz, 1H), 7.57 (d, J=3.6 Hz, 1H), 7.34 (d, J=8.3 Hz, 2H), 7.19 (d, J=3.6 Hz, 1H), 2.64 (t, J=7.7 Hz, 2H), 1.60 (p, 2H), 1.35 (h, J=7.3 Hz, 2H), 0.93 (t, J=7.3 Hz, 3H). ¹³C NMR (101 MHz, DMSO-d₆) δ 171.77, 171.16, 165.59, 156.05, 152.01, 143.42, 141.87, 135.36, 133.98, 129.44, 125.57, 124.59, 124.24, 124.19, 122.56, 108.80, 35.11, 33.43, 22.22, 14.25. HRMS (ESI) calcd for C₂₄H₂₁ClNO₃ [M+H]⁺, 406.1132; found 406.12045.

### 7-bromo-2-(5-(4-butylphenyl)furan-2-yl)quinoline-4-carboxylic acid (10)

¹H NMR (400 MHz, DMSO-d₆) δ 8.60 (d, J=9.1 Hz, 1H), 8.40 (s, 1H), 8.29 (d, J=2.1 Hz, 1H), 7.85-7.77 (m, 3H), 7.58 (d, J=3.6 Hz, 1H), 7.36-7.30 (m, 2H), 7.19 (d, J=3.6 Hz, 1H), 2.64 (t, J=7.7 Hz, 2H), 1.60 (p, 2H), 1.35 (h, J=7.3 Hz, 2H), 0.93 (t, J=7.3 Hz, 3H). ¹³C NMR (126 MHz, DMSO-d₆) δ 169.50, 166.72, 163.77, 156.23, 151.82, 149.55, 143.53, 133.96, 131.48, 130.85, 129.49, 127.59, 124.65, 124.31, 122.70, 118.95, 114.99, 112.56, 109.83, 108.86, 107.45, 22.09, 14.10. HRMS (ESI) calcd for C₂₄H₂₁BrNO₃ [M+H]⁺, 450.0627; found 450.07013.

### 2-(5-(4-butylphenyl)furan-2-yl)-8-chloroquinoline-4-carboxylic acid (11)

¹H NMR (400 MHz, DMSO-d₆) δ 8.56 (dd, J=1.3, 8.5 Hz, 1H), 8.42 (s, 1H), 8.02 (dd, J=1.3, 7.5 Hz, 1H), 7.87-7.80 (m, 2H), 7.66-7.55 (m, 2H), 7.38-7.31 (m, 2H), 7.21 (d, J=3.6 Hz, 1H), 2.64 (t, J=7.7 Hz, 2H), 1.60 (p, 2H), 1.35 (h, J=7.3 Hz, 2H), 0.93 (t, J=7.3 Hz, 3H). ¹³C NMR (101 MHz, DMSO-d₆) δ 167.65, 156.08, 151.94, 151.31, 148.72, 143.42, 133.44, 132.93, 130.96, 129.46, 127.72, 127.59, 125.48, 125.19, 124.58, 114.57, 108.83, 35.11, 33.41, 22.21, 14.25. HRMS (ESI) calcd for C₂₄H₂₁ClNO₃ [M+H]⁺, 406.1132; found 406.12057.

### 8-bromo-2-(5-(4-butylphenyl)furan-2-yl)quinoline-4-carboxylic acid (12)

¹H NMR (400 MHz, DMSO-d₆) δ 8.60 (dd, J=1.3, 8.5 Hz, 1H), 8.43 (s, 1H), 8.23 (dd, J=1.2, 7.5 Hz, 1H), 7.88-7.81 (m, 2H), 7.62-7.52 (m, 2H), 7.35 (d, J=8.2 Hz, 2H), 7.22 (d, J=3.6 Hz, 1H), 2.65 (t, J=7.7 Hz, 2H), 1.60 (p, 2H), 1.35 (h, J=7.3 Hz, 2H), 0.93 (t, J=7.3 Hz, 3H). ¹³C NMR (126 MHz, DMSO-d₆) δ 166.44, 160.74, 158.08, 153.50, 141.93, 138.26, 132.86, 129.52, 127.63, 126.21, 125.37, 124.62, 123.11, 120.36, 119.11, 111.78, 108.86, 35.02, 33.30, 24.87, 22.14, 16.74, 14.10. HRMS (ESI) calcd for C₂₄H₂₁BrNO₃ [M+H]⁺, 450.0627; found 450.06985.

### 2-(5-(4-butylphenyl)furan-2-yl)-6,7-difluoroquinoline-4-carboxylic acid (13)

¹H NMR (400 MHz, DMSO-d₆) δ 8.69 (dd, J=9.1, 12.7 Hz, 1H), 8.44 (s, 1H), 8.11 (dd, J=8.1, 11.6 Hz, 1H), 7.82 (d, 2H), 7.56 (d, J=3.6 Hz, 1H), 7.35 (d, 2H), 7.19 (d, J=3.6 Hz, 1H), 2.65 (t, J=7.7 Hz, 2H), 1.60 (p, 2H), 1.35 (h, J=7.3 Hz, 2H), 0.93 (t, J=7.3 Hz, 3H). HRMS (ESI) calcd for C₂₄H₂₀F₂NO₃ [M+H]⁺, 408.1333; found 408.14047.

### 2-(5-(4-butylphenyl)furan-2-yl)-6,7-dichloroquinoline-4-carboxylic acid (14)

¹H NMR (400 MHz, DMSO-d₆) δ 8.99 (s, 1H), 8.38 (s, 1H), 8.30 (s, 1H), 7.85-7.76 (m, 2H), 7.56 (d, J=3.6 Hz, 1H), 7.35-7.29 (m, 2H), 7.18 (d, J=3.6 Hz, 1H), 2.64 (t, J=7.7 Hz, 2H), 1.60 (p, 2H), 1.34 (h, 2H), 0.93 (t, J=7.3 Hz, 3H). ¹³C NMR (126 MHz, DMSO-d₆) δ 156.32, 155.57, 154.21, 153.17, 151.81, 149.86, 148.04, 143.90, 143.54, 140.10, 134.76, 133.39, 130.12, 129.49, 127.53, 126.02, 124.64, 109.81, 38.91, 34.99, 33.29, 22.08, 14.09, 9.28. HRMS (ESI) calcd for C₂₄H₂₀C₁₂NO₃ [M+H]⁺, 440.0742; found 440.08140.

### 2-(5-(4-butylphenyl)furan-2-yl)-6,8-dichloroquinoline-4-carboxylic acid (15)

¹H NMR (400 MHz, DMSO-d₆) δ 8.71 (d, J=2.3 Hz, 1H), 8.49 (s, 1H), 8.16 (d, J=2.3 Hz, 1H), 7.83 (d, J=8.2 Hz, 2H), 7.61 (d, J=3.6 Hz, 1H), 7.36-7.31 (m, 2H), 7.21 (d, J=3.6 Hz, 1H), 2.64 (t, J=7.7 Hz, 2H), 1.60 (p, J=6.4, 7.8 Hz, 2H), 1.35 (h, J=7.3 Hz, 2H), 0.93 (t, J=7.4 Hz, 3H). ¹³C NMR (126 MHz, DMSO-d₆) δ 174.34, 167.20, 156.43, 151.73, 147.06, 144.46, 140.42, 137.91, 136.29, 134.49, 133.78, 130.93, 129.54, 127.50, 125.50, 124.66, 124.46, 121.29, 109.80, 34.99, 33.29, 22.03, 14.06. HRMS (ESI) calcd for C₂₄H₂₀Cl₂NO₃ [M+H]⁺, 440.0742; found 440.08151.

### 2-(5-(4-butylphenyl)furan-2-yl)-6-methylquinoline-4-carboxylic acid (16)

¹H NMR (400 MHz, DMSO-d₆) δ 8.39 (s, 1H), 8.32 (s, 1H), 8.00 (d, J=8.6 Hz, 1H), 7.84-7.76 (m, 2H), 7.68 (dd, J=2.0, 8.7 Hz, 1H), 7.50 (d, J=3.6 Hz, 1H), 7.33 (d, J=8.1 Hz, 2H), 7.16 (d, J=3.6 Hz, 1H), 2.64 (t, J=7.7 Hz, 2H), 2.55 (s, 3H), 1.60 (p, J=6.4, 7.8 Hz, 2H), 1.35 (h, J=7.3 Hz, 2H), 0.93 (t, J=7.3 Hz, 3H). ¹³C NMR (126 MHz, DMSO-d₆) δ 170.31, 168.15, 160.76, 157.84, 155.58, 152.29, 147.64, 143.32, 130.25, 130.03, 129.48, 127.74, 126.83, 126.32, 125.41, 125.30, 125.21, 124.86, 124.50, 108.68, 34.98, 33.32, 22.07, 21.91, 14.10. HRMS (ESI) calcd for C₂₅H₂₄NO₃ [M+H]⁺, 386.1678; found 386.17500.

### 2-(5-(4-butylphenyl)furan-2-yl)-6-ethylquinoline-4-carboxylic acid (17)

¹H NMR (400 MHz, DMSO-d₆) δ 8.41 (d, J=1.8 Hz, 1H), 8.32 (s, 1H), 8.02 (d, J=8.6 Hz, 1H), 7.84-7.79 (m, 2H), 7.73 (dd, J=2.0, 8.8 Hz, 1H), 7.50 (d, J=3.6 Hz, 1H), 7.37-7.30 (m, 2H), 7.17 (d, J=3.6 Hz, 1H), 2.85 (q, J=7.6 Hz, 2H), 2.64 (t, J=7.7 Hz, 2H), 1.60 (p, 2H), 1.39-1.26 (m, 5H), 0.93 (t, J=7.3 Hz, 3H). HRMS (ESI) calcd for C₂₆H₂₆NO₃ [M+H]⁺, 400.1834; found 400.19063.

### 2-(5-(4-butylphenyl)furan-2-yl)-6-isopropylquinoline-4-carboxylic acid (18)

¹H NMR (400 MHz, DMSO-d₆) δ 8.44 (d, J=1.9 Hz, 1H), 8.33 (s, 1H), 8.04 (d, J=8.7 Hz, 1H), 7.84-7.76 (m, 3H), 7.50 (d, J=3.6 Hz, 1H), 7.33 (d, J=8.0 Hz, 2H), 7.17 (d, J=3.6 Hz, 1H), 3.13 (p, J=6.8 Hz, 1H), 2.64 (t, J=7.7 Hz, 2H), 1.61 (p, 2H), 1.40-1.27 (m, 8H), 0.93 (t, J=7.3 Hz, 3H). ¹³C NMR (126 MHz, DMSO-d₆) δ 162.58, 155.55, 152.33, 150.68, 147.93, 147.72, 144.57, 143.24, 133.85, 129.72, 129.46, 127.76, 124.50, 121.40, 115.65, 113.60, 109.79, 108.63, 34.99, 34.13, 33.32, 23.99, 22.08, 14.10. HRMS (ESI) calcd for C₂₇H₂₇NO₃ [M+H]⁺, 414.1991; found 414.20633.

### 2-(5-(4-butylphenyl)furan-2-yl)-6-phenylquinoline-4-carboxylic acid (19)

¹H NMR (400 MHz, DMSO-d₆) δ 8.91 (d, J=1.8 Hz, 1H), 8.43 (s, 1H), 8.21-8.12 (m, 2H), 7.82 (t, J=7.9 Hz, 4H), 7.60-7.52 (m, 3H), 7.46 (t, J=7.3 Hz, 1H), 7.35 (d, J=8.0 Hz, 2H), 7.20 (d, J=3.6 Hz, 1H), 2.65 (t, J=7.7 Hz, 2H), 1.61 (p, J=6.3, 7.9 Hz, 2H), 1.35 (h, J=7.4 Hz, 2H), 0.93 (t, J=7.3 Hz, 3H). ¹³C NMR (126 MHz, DMSO-d₆) δ 167.93, 155.86, 153.83, 152.16, 148.48, 145.50, 143.35, 141.66, 140.10, 140.05, 139.42, 135.58, 133.69, 131.06, 130.43, 130.40, 129.75, 129.50, 127.77, 127.62, 124.58, 124.14, 119.01, 117.68, 95.27, 35.00, 33.32, 22.09, 14.10. HRMS (ESI) calcd for C₃₀H₂₆NO₃ [M+H]⁺, 448.1834; found 448.19066.

### 2-(5-(4-butylphenyl)furan-2-yl)-6,8-dimethylquinoline-4-carboxylic acid (20)

¹H NMR (500 MHz, Chloroform-d) δ 8.29 (s, 1H), 8.19 (s, 1H), 7.84-7.79 (m, 2H), 7.55 (d, J=1.8 Hz, 1H), 7.48 (d, J=3.6 Hz, 1H), 7.36-7.31 (m, 2H), 7.17 (d, J=3.5 Hz, 1H), 2.78 (s, 3H), 2.64 (t, J=7.7 Hz, 2H), 2.49 (s, 3H), 1.60 (p, 2H), 1.34 (h, J=7.4 Hz, 2H), 0.92 (t, J=7.3 Hz, 3H). ¹³C NMR (126 MHz, Chloroform-d) δ 163.04, 159.63, 158.80, 155.42, 140.38, 140.05, 139.61, 138.69, 137.46, 136.56, 130.26, 127.80, 127.33, 126.59, 126.27, 125.62, 124.47, 122.72, 122.10, 120.01, 109.76, 33.30, 22.08, 18.12, 14.10. HRMS (ESI) calcd for C₂₆H₂₆NO₃ [M+H]⁺, 400.1834; found 400.19099.

### 2-(5-(4-butylphenyl)furan-2-yl)-6-methoxyquinoline-4-carboxylic acid (21)

¹H NMR (500 MHz, DMSO-d₆) δ 8.37 (s, 1H), 8.14 (d, J=2.8 Hz, 1H), 8.03 (d, J=9.2 Hz, 1H), 7.80 (d, J=8.3 Hz, 2H), 7.51 (dd, J=2.9, 9.2 Hz, 1H), 7.44 (d, J=3.6 Hz, 1H), 7.33 (d, J=8.2 Hz, 2H), 7.15 (d, J=3.5 Hz, 1H), 3.93 (s, 3H), 2.64 (t, J=7.7 Hz, 2H), 1.59 (p, 2H), 1.34 (h, J=7.3 Hz, 2H), 0.92 (t, J=7.4 Hz, 3H). ¹³C NMR (126 MHz, DMSO-d₆) δ 168.11, 159.48, 159.01, 158.91, 158.13, 156.45, 155.29, 154.61, 153.27, 152.40, 149.48, 147.30, 143.17, 139.33, 134.22, 134.10, 132.87, 129.46, 127.03, 124.45, 120.90, 112.92, 109.81, 55.85, 22.08, 14.10. HRMS (ESI) calcd for C₂₅H₂₄NO₄ [M+H]⁺, 402.1627; found 402.17019.

### 2-(5-(4-butylphenyl)furan-2-yl)-7-methoxyquinoline-4-carboxylic acid (22)

¹H NMR (400 MHz, DMSO-d₆) δ 8.54 (d, J=9.3 Hz, 1H), 8.22 (s, 1H), 7.82 (d, J=8.3 Hz, 2H), 7.51 (d, J=3.6 Hz, 1H), 7.48 (d, J=2.7 Hz, 1H), 7.36-7.29 (m, 3H), 7.17 (d, J=3.6 Hz, 1H), 3.98 (s, 3H), 2.65 (t, J=7.7 Hz, 2H), 1.61 (p, 2H), 1.35 (h, J=7.3 Hz, 2H), 0.93 (t, J=7.3 Hz, 3H). ¹³C NMR (126 MHz, DMSO-d₆) δ 170.31, 168.15, 160.76, 157.84, 155.58, 152.29, 147.64, 143.32, 130.25, 130.03, 129.48, 127.74, 126.83, 126.32, 125.41, 125.30, 125.21, 124.86, 124.50, 108.68, 34.98, 33.32, 22.07, 21.91, 14.10. HRMS (ESI) calcd for C₂₅H₂₄NO₄ [M+H]⁺, 402.1627; found 402.17014.

### 2-(5-(4-butylphenyl)furan-2-yl)-6-(trifluoromethoxy)quinoline-4-carboxylic acid (23)

¹H NMR (400 MHz, DMSO-d₆) δ 8.71 (dd, J=1.3, 2.7 Hz, 1H), 8.49 (s, 1H), 8.22 (d, J=9.2 Hz, 1H), 7.86-7.80 (m, 3H), 7.60 (d, J=3.6 Hz, 1H), 7.35 (d, J=8.3 Hz, 2H), 7.20 (d, J=3.6 Hz, 1H), 2.65 (t, J=7.7 Hz, 2H), 1.61 (p, J=7.4, 8.9 Hz, 2H), 1.35 (h, J=7.3, 14.6 Hz, 2H), 0.93 (t, J=7.4 Hz, 3H). ¹³C NMR (126 MHz, DMSO-d₆) δ 167.44, 156.20, 151.78, 147.48, 146.29, 141.86, 140.47, 138.82, 138.20, 134.12, 133.00, 132.29, 130.55, 129.50, 127.66, 125.31, 124.63, 124.29, 119.50, 109.68, 108.80, 35.07, 33.31, 22.08, 14.09. HRMS (ESI) calcd for C₂₅H₂₁F₃NO₄ [M+H]⁺, 456.1344; found 456.14191.

### 6-amino-2-(5-(4-butylphenyl)furan-2-yl)quinoline-4-carboxylic acid (24)

¹H NMR (400 MHz, DMSO-d₆) δ 8.19 (s, 1H), 7.81-7.74 (m, 3H), 7.65 (d, J=2.5 Hz, 1H), 7.32 (d, J=8.1 Hz, 2H), 7.27 (d, J=3.5 Hz, 1H), 7.23 (dd, J=2.5, 9.0 Hz, 1H), 7.10 (d, J=3.5 Hz, 1H), 6.00 (s, 2H), 2.63 (t, J=7.7 Hz, 2H), 1.60 (p, J=6.4, 7.9 Hz, 2H), 1.35 (h, J=7.4 Hz, 2H), 0.93 (t, J=7.3 Hz, 3H). ¹³C NMR (126 MHz, DMSO-d₆) δ 168.60, 154.43, 146.07, 143.50, 130.82, 129.40, 127.89, 125.07, 124.20, 122.86, 122.12, 121.59, 121.28, 118.38, 116.90, 116.46, 106.38, 103.30, 72.65, 35.03, 33.34, 22.07, 14.10. HRMS (ESI) calcd for C₂₄H₂₃N₂O₃ [M+H]⁺, 387.1630; found 387.17051.

### 6-acetamido-2-(5-(4-butylphenyl)furan-2-yl)quinoline-4-carboxylic acid (25)

¹H NMR (500 MHz, DMSO-d₆) δ 10.39 (s, 1H), 8.87 (d, J=2.3 Hz, 1H), 8.30 (s, 1H), 8.10 (dd, J=2.3, 9.2 Hz, 1H), 8.03 (d, J=9.1 Hz, 1H), 7.81 (d, J=8.1 Hz, 2H), 7.47 (d, J=3.6 Hz, 1H), 7.34 (d, J=8.1 Hz, 2H), 7.16 (d, J=3.6 Hz, 1H), 2.64 (t, J=6.4, 8.8 Hz, 2H), 2.13 (s, 3H), 1.60 (p, J=6.4, 7.8 Hz, 2H), 1.35 (h, J=7.3 Hz, 2H), 0.93 (t, J=7.4 Hz, 3H). ¹³C NMR (126 MHz, DMSO-d₆) δ 163.41, 159.64, 150.89, 147.18, 140.92, 139.20, 138.91, 135.76, 132.47, 131.65, 130.19, 124.49, 123.50, 121.72, 119.54, 117.80, 117.04, 111.02, 107.49, 105.46, 72.68, 33.18, 27.65, 22.12, 12.59, −3.71. HRMS (ESI) calcd for C₂₆H₂₅N₂O₄ [M+H]⁺, 429.1736; found 429.18117.

### 2-(5-(4-butylphenyl)furan-2-yl)-7-(trifluoromethyl)quinoline-4-carboxylic acid (26)

¹H NMR (400 MHz, DMSO-d₆) δ 8.90-8.84 (m, 1H), 8.53 (s, 1H), 8.41 (dt, J=0.9, 1.8 Hz, 1H), 7.93 (dd, J=2.0, 9.0 Hz, 1H), 7.87-7.81 (m, 2H), 7.65 (d, J=3.6 Hz, 1H), 7.35 (d, J=8.3 Hz, 2H), 7.22 (d, J=3.6 Hz, 1H), 2.65 (t, J=7.7 Hz, 2H), 1.61 (p, J=7.4, 8.9 Hz, 2H), 1.35 (h, J=7.3 Hz, 2H), 0.93 (t, J=7.3 Hz, 3H). ¹³C NMR (126 MHz, DMSO-d₆) δ 167.43, 162.84, 160.79, 156.43, 151.58, 148.15, 145.79, 143.73, 137.52, 136.17, 134.04, 129.51, 124.72, 118.21, 116.99, 114.80, 111.57, 109.04, 60.72, 53.60, 35.00, 33.31, 22.09, 14.10. HRMS (ESI) calcd for C₂₅H₂₁F₃NO₃ [M+H]⁺, 440.1395; found 440.14691.

### 2-(5-(4-butylphenyl)furan-2-yl)-8-(trifluoromethyl)quinoline-4-carboxylic acid (27)

¹H NMR (400 MHz, DMSO-d₆) δ 8.89 (dd, J=1.3, 8.7 Hz, 1H), 8.49 (s, 1H), 8.26 (d, J=7.3 Hz, 1H), 7.87-7.81 (m, 2H), 7.81-7.75 (m, 1H), 7.55 (d, J=3.6 Hz, 1H), 7.38-7.32 (m, 2H), 7.22 (d, J=3.6 Hz, 1H), 2.65 (t, J=7.7 Hz, 2H), 1.60 (p, 2H), 1.35 (h, J=7.3 Hz, 2H), 0.93 (t, J=7.3 Hz, 3H). ¹³C NMR (126 MHz, DMSO-d₆) δ 167.64, 156.48, 154.92, 151.84, 143.59, 141.98, 139.96, 138.59, 129.53, 127.62, 126.72, 125.83, 124.62, 124.22, 121.60, 115.22, 111.68, 109.81, 108.88, 100.02, 34.99, 33.29, 22.06, 14.10. HRMS (ESI) calcd for C₂₅H₂₁F₃NO₃ [M+H]⁺, 440.1395; found 440.14700.

### 2-(5-(4-butylphenyl)furan-2-yl)-6-nitroquinoline-4-carboxylic acid (compound 28)

¹H NMR (400 MHz, DMSO-d₆) δ 9.66 (d, J=2.6 Hz, 1H), 8.56 (s, 1H), 8.51 (dd, J=2.6, 9.3 Hz, 1H), 8.25 (d, J=9.2 Hz, 1H), 7.84 (d, 2H), 7.74 (d, J=3.7 Hz, 1H), 7.36 (d, 2H), 7.25 (d, J=3.7 Hz, 1H), 2.65 (t, J=7.7 Hz, 2H), 1.61 (p, 2H), 1.35 (h, J=7.3 Hz, 2H), 0.93 (t, J=7.3 Hz, 3H). ¹³C NMR (126 MHz, DMSO-d₆) δ 167.06, 157.10, 153.68, 151.55, 151.36, 145.69, 143.88, 139.76, 131.39, 129.53, 127.37, 124.83, 124.19, 122.82, 120.73, 101.53, 35.01, 33.28, 22.08, 14.09. HRMS (ESI) calcd for C₂₄H₂₁N₂O₅ [M+H]⁺, 417.1372; found 417.14449.

### 2-(5-(4-butylphenyl)furan-2-yl)-8-nitroquinoline-4-carboxylic acid (30)

¹H NMR (400 MHz, DMSO-d₆) δ 8.86 (dd, J=1.3, 8.6 Hz, 1H), 8.52 (s, 1H), 8.32 (dd, J=1.3, 7.6 Hz, 1H), 7.86-7.76 (m, 3H), 7.56 (d, J=3.6 Hz, 1H), 7.35 (d, J=8.1 Hz, 2H), 7.22 (d, J=3.6 Hz, 1H), 2.65 (t, J=7.6 Hz, 2H), 1.61 (p, 2H), 1.34 (h, J=7.3 Hz, 2H), 0.93 (t, J=7.3 Hz, 3H). ¹³C NMR (126 MHz, DMSO-d₆) δ 167.30, 163.02, 156.75, 153.84, 151.40, 149.92, 148.55, 144.16, 139.94, 138.64, 135.04, 129.55, 127.34, 124.75, 114.50, 109.29, 91.76, 77.75, 65.22, 35.00, 33.27, 22.06, 14.09, 2.71. HRMS (ESI) calcd for C₂₄H₂₁N₂O₅ [M+H]⁺, 417.1372; found 417.14440.

### 2-(5-(4-butylphenyl)thiophen-2-yl)-6-nitroquinoline-4-carboxylic acid (31)

¹H NMR (400 MHz, DMSO) δ 9.72 (d, J=2.7 Hz, 1H), 8.43 (s, 1H), 8.42 (dd, J=9.2, 2.7 Hz, 1H), 8.17 (d, J=4.0 Hz, 1H), 8.12 (d, J=9.2 Hz, 1H), 7.72 (d, J=8.0 Hz, 2H), 7.62 (d, J=4.1 Hz, 1H), 7.30 (d, J=8.0 Hz, 2H), 2.63 (t, J=7.6 Hz, 2H), 1.59 (p, 2H), 1.34 (h, J=7.4 Hz, 2H), 0.93 (t, J=7.3 Hz, 3H). ¹³C NMR (126 MHz, DMSO) δ 167.29, 155.29, 150.91, 148.87, 145.80, 144.99, 143.57, 142.66, 131.23, 130.85, 130.63, 129.64, 126.09, 125.38, 124.40, 123.59, 118.95, 34.99, 33.40, 22.20, 14.25. HRMS (ESI) calcd for C₂₄H₂₁N₂O₄S [M+H]⁺ 433.1144; found 433.1215.

### 2-(5-(4-butylphenyl)-1H-pyrrol-2-yl)-6-nitroquinoline-4-carboxylic acid (32)

¹H NMR (400 MHz, DMSO) δ 12.08 (s, 1H), 9.64 (d, J=2.6 Hz, 1H), 8.49 (s, 1H) 8.46 (dd, J=9.2, 2.7 Hz, 1H), 8.12 (d, J=9.2 Hz, 1H), 7.76-7.45 (m, 4H), 7.17 (d, J=8.0 Hz, 2H), 2.58 (t, J=7.6 Hz, 2H), 1.57 (p, 2H), 1.34 (h, J=7.3 Hz, 2H), 0.92 (t, J=7.3 Hz, 3H). ¹³C NMR (126 MHz, DMSO) δ 187.65, 172.54, 167.36, 153.40, 151.41, 144.71, 140.17, 132.64, 131.84, 130.47, 129.05, 126.74, 125.52, 123.87, 122.41, 121.90, 114.03, 110.52, 34.96, 33.64, 22.21, 14.26. HRMS (ESI) calcd for C₂₄H₂₂N₂O₄[M+H]⁺ 416.1532; found 416.1605.

### 2-(4′-butyl-[1,1′-biphenyl]-3-yl)-6-nitroquinoline-4-carboxylic acid (33)

¹H NMR (400 MHz, DMSO) δ 9.84 (d, J=2.7 Hz, 1H), 8.56-8.51 (m, 2H), 8.47 (dd, J=9.2, 2.7 Hz, 1H), 8.28 (dd, J=8.9, 2.3 Hz, 2H), 7.84 (d, J=8.0 Hz, 1H), 7.72 (d, J=8.0 Hz, 2H), 7.67 (t, J=7.7 Hz, 1H), 7.35 (d, J=8.0 Hz, 2H), 2.66 (t, J=7.7 Hz, 2H), 1.61 (p, J=7.6 Hz, 2H), 1.36 (h, J=7.3 Hz, 2H), 0.94 (t, J=7.3 Hz, 3H). ¹³C NMR (126 MHz, DMSO) δ 167.77, 164.86, 159.84, 150.33, 145.50, 142.41, 141.44, 139.08, 137.88, 131.61, 129.65, 129.14, 127.84, 127.46, 124.74, 124.67, 124.14, 123.53, 120.21, 35.13, 33.85, 22.30, 14.67. HRMS (ESI) calcd for C₂₆H₂₃N₂O₄ [M+H]⁺ 427.1580; found 427.1653.

### 2-(6-(4-butylphenyl)pyridin-2-yl)-6-nitroquinoline-4-carboxylic acid (34)

¹H NMR (400 MHz, DMSO) δ 9.88 (d, J=2.7 Hz, 1H), 9.14 (s, 1H), 8.59 (t, J=4.4 Hz, 1H), 8.51 (dd, J=9.2, 2.7 Hz, 1H), 8.34 (d, J=9.2 Hz, 1H), 8.17 (d, J=8.1 Hz, 2H), 8.15-8.12 (m, 2H), 7.41 (d, J=8.1 Hz, 2H), 2.68 (t, J=7.7 Hz, 2H), 1.64 (p, J=7.6 Hz, 2H), 1.37 (h, J=7.4 Hz, 2H), 0.94 (t, J=7.3 Hz, 3H). ¹³C NMR (126 MHz, DMSO) δ 167.53, 159.18, 156.47, 154.34, 150.63, 149.19, 145.88, 144.42, 139.15, 136.21, 134.77, 131.85, 129.39, 127.09, 124.44, 124.38, 123.40, 122.06, 120.53, 116.81, 107.38, 35.07, 33.45, 22.24, 14.27. HRMS (ESI) calcd for C₂₅H₂₂N₃O₄ [M+H]⁺ 428.1532; found 428.1603.

### 2-(5-(4-butylphenyl)pyridin-3-yl)-6-nitroquinoline-4-carboxylic acid (35)

¹H NMR (400 MHz, DMSO) δ 9.75 (d, J=2.6 Hz, 1H), 9.45 (d, J=2.1 Hz, 1H), 9.04 (d, J=2.2 Hz, 1H), 8.86 (t, J=2.2 Hz, 1H), 8.71 (s, 1H), 8.52 (dd, J=9.3, 2.7 Hz, 1H), 8.35 (d, J=9.3 Hz, 1H), 7.82 (d, J=8.0 Hz, 2H), 7.38 (d, J=8.1 Hz, 2H), 2.68 (t, J=7.6 Hz, 2H), 1.63 (p, J=7.6 Hz, 2H), 1.36 (h, J=7.4 Hz, 2H), 0.94 (t, J=7.3 Hz, 3H). ¹³C NMR (126 MHz, DMSO) δ 167.47, 157.65, 150.94, 149.43, 147.61, 146.10, 143.39, 139.69, 132.97, 129.65, 127.46, 124.14, 123.83, 120.51, 113.49, 101.56, 61.93, 40.57, 39.36, 35.13, 33.85, 22.00, 14.37. HRMS (ESI) calcd for C₂₅H₂₂N₃O₄ [M+H]⁺ 428.1532; found 428.1604.

### 2-(4′-butyl-[1,1′-biphenyl]-4-yl)-6-nitroquinoline-4-carboxylic acid (36)

¹H NMR (400 MHz, DMSO) δ 9.98 (d, J=2.7 Hz, 1H), 8.43-8.35 (m, 4H), 8.19 (d, J=9.2 Hz, 1H), 7.88 (d, J=8.0 Hz, 2H), 7.71 (d, J=8.0 Hz, 2H), 7.34 (d, J=8.1 Hz, 2H), 2.66 (t, J=7.7 Hz, 2H), 1.62 (p, J=7.6 Hz, 2H), 1.36 (h, J=7.4 Hz, 2H), 0.94 (t, J=7.3 Hz, 3H). ¹³C NMR (126 MHz, DMSO) δ 167.48, 165.47, 159.27, 152.29, 150.98, 144.70, 142.74, 142.22, 137.33, 131.14, 129.49, 127.43, 127.09, 125.78, 124.66, 122.68, 118.67, 34.94, 33.53, 22.24, 14.27. HRMS (ESI) calcd for C₂₆H₂₃N₂O₄ [M+H]⁺ 427.1580; found 427.1652.

### 2-(6-(4-butylphenyl)pyridin-3-yl)-6-nitroquinoline-4-carboxylic acid (37)

¹H NMR (400 MHz, DMSO) δ 9.89 (d, J=2.7 Hz, 1H), 9.54 (d, J=2.4 Hz, 1H), 8.74 (dd, J=8.4, 2.4 Hz, 1H), 8.45 (dd, J=9.2, 2.8 Hz, 1H), 8.44 (s, 1H), 8.25 (d, J=9.3 Hz, 1H), 8.18-8.09 (m, 3H), 7.37 (d, J=8.1 Hz, 2H), 2.68 (t, J=7.7 Hz, 2H), 1.63 (p, J=7.5 Hz, 2H), 1.36 (h, J=7.4 Hz, 2H), 0.94 (t, J=7.3 Hz, 3H). ¹³C NMR (126 MHz, DMSO) δ 167.47, 161.35, 157.96, 150.94, 149.12, 145.50, 136.67, 136.06, 132.16, 131.76, 129.35, 126.55, 125.04, 123.23, 120.51, 119.45, 119.23, 115.30, 35.13, 33.55, 22.60, 14.07. HRMS (ESI) calcd for C₂₅H₂₂N₃O₄ [M+H]⁺ 428.1532; found 428.1604.

### 6-nitro-2-(5-phenylfuran-2-yl)quinoline-4-carboxylic acid (38)

¹H NMR (400 MHz, DMSO-d₆) δ 9.71 (d, J=2.7 Hz, 1H), 8.49 (s, 1H), 8.47 (dd, J=9.2, 2.4 Hz, 1H), 8.22 (d, J=9.4 Hz, 1H), 7.93 (d, J=8.0 Hz, 2H), 7.70 (d, J=3.6 Hz, 1H), 7.53 (t, J=7.6 Hz, 2H), 7.41 (t, J=7.4 Hz, 1H), 7.30 (d, J=3.7 Hz, 1H). ¹³C NMR (126 MHz, DMSO) δ 167.17, 156.45, 152.22, 151.62, 150.94, 145.50, 145.20, 131.46, 129.95, 129.35, 125.04, 124.14, 123.53, 120.21, 119.83, 116.21, 110.10. HRMS (ESI) calcd for C₂₀H₁₃N₂O₅ [M+H]⁺ 361.0746; found 361.0819.

### 6-nitro-2-(5-(4-propylphenyl)furan-2-yl)quinoline-4-carboxylic acid (39)

¹H NMR (400 MHz, DMSO-d₆) δ 9.70 (d, J=2.6 Hz, 1H), 8.50 (s, 1H), 8.48 (dd, J=9.2, 2.4 Hz, 1H), 8.22 (d, J=9.3 Hz, 1H), 7.87-7.82 (m, 2H), 7.70 (d, J=3.7 Hz, 1H), 7.35 (d, J=8.0 Hz 2H), 7.24 (d, J=3.6 Hz, 1H), 2.63 (t, J=7.5 Hz, 2H), 1.65 (h, 2H), 0.94 (t, J=7.3 Hz, 3H). ¹³C NMR (126 MHz, DMSO) δ 167.47, 156.75, 151.62, 151.21, 145.50, 143.99, 131.22, 129.65, 127.46, 124.74, 120.51, 116.81, 116.51, 109.19, 40.27, 39.96, 37.85, 37.53, 24.41, 14.07. HRMS (ESI) calcd for C₂₃H₁₉N₂O₅ [M+H]⁺ 403.1216; found 403.1288.

### 6-nitro-2-(5-(4-pentylphenyl)furan-2-yl)quinoline-4-carboxylic acid (40)

¹H NMR (400 MHz, DMSO-d₆) δ 9.74 (d, J=2.6 Hz, 1H), 8.47 (dd, J=9.2, 2.4 Hz, 1H), 8.45 (s, 1H), 8.21 (d, J=9.3 Hz, 1H), 7.84 (d, J=8.0 Hz, 2H), 7.69 (d, J=3.6 Hz, 1H), 7.35 (d, J=8.0 Hz, 2H), 7.23 (d, J=3.6 Hz, 1H), 2.64 (t, J=7.7 Hz, 2H), 1.63 (p, J=7.5 Hz, 2H), 1.41-1.26 (m, 5H), 0.89 (t, J=6.8 Hz, 3H). ¹³C NMR (126 MHz, DMSO) δ 167.10, 163.55, 156.74, 151.86, 151.39, 145.26, 143.73, 135.10, 131.10, 129.51, 127.46, 123.78, 116.25, 109.14, 106.77, 40.58, 38.15, 35.41, 31.36, 30.93, 22.42, 14.41. HRMS (ESI) calcd for C₂₅H₂₃N₂O₅ [M+H]⁺ 431.1529; found 431.1601.

### 6-nitro-2-(5-(4-propoxyphenyl)furan-2-yl)quinoline-4-carboxylic acid (41)

¹H NMR (400 MHz, DMSO) δ 9.72 (d, J=2.7 Hz, 1H), 8.46 (dd, J=9.3, 2.7 Hz, 1H), 8.44 (s, 1H), 8.19 (d, J=9.3 Hz, 1H), 7.88-7.81 (m, 2H), 7.66 (d, J=3.6 Hz, 1H), 7.14 (d, J=3.7 Hz, 1H), 7.08 (d, J=8.0 Hz, 2H), 4.01 (t, J=6.5 Hz, 2H), 1.78 (h, J=7.1 Hz, 2H), 1.02 (t, J=7.4 Hz, 3H). ¹³C NMR (126 MHz, DMSO) δ 167.16, 159.65, 156.82, 151.41, 145.19, 141.80, 132.67, 131.04, 126.39, 123.91, 123.21, 122.51, 119.70, 119.53, 116.47, 115.52, 108.12, 69.59, 22.49, 10.87. HRMS (ESI) calcd for C₂₃H₁₉N₂O₆ [M+H]⁺ 419.1165; found 419.1236.

### 6-nitro-2-(5-(3-propoxyphenyl)furan-2-yl)quinoline-4-carboxylic acid (42)

¹H NMR (400 MHz, DMSO) δ 9.86 (d, J=2.7 Hz, 1H), 8.39 (dd, J=9.2, 2.7 Hz, 1H), 8.27 (s, 1H), 8.13 (d, J=9.3 Hz, 1H), 7.60 (d, J=3.6 Hz, 1H), 7.50 (d, J=8.0, 1H), 7.46-7.39 (m, 2H), 7.30 (d, J=3.7 Hz, 1H), 6.97 (dd, J=8.3, 2.5, Hz, 1H), 4.04 (t, J=6.5 Hz, 2H), 1.80 (h, J=7.1 Hz, 2H), 1.04 (t, J=7.4, 3H). ¹³C NMR (126 MHz, DMSO) δ 167.41, 159.68, 155.81, 152.64, 151.42, 151.11, 144.67, 131.26, 130.79, 130.69, 125.28, 124.19, 123.14, 117.86, 116.92, 115.28, 115.07, 110.52, 109.94, 69.59, 22.55, 10.94. HRMS (ESI) calcd for C₂₃H₁₉N₂O₆ [M+H]⁺ 419.1165; found 419.1237.

### 6-nitro-2-(5-(2-propoxyphenyl)furan-2-yl)quinoline-4-carboxylic acid (43)

¹H NMR (400 MHz, DMSO) δ 9.82 (d, J=2.7 Hz, 1H), 8.41 (dd, J=9.3, 2.7 Hz, 1H), 8.34 (s, 1H), 8.14 (d, J=9.2 Hz, 1H), 8.02 (dd, J=7.8, 1.7 Hz, 1H), 7.62 (d, J=3.6 Hz, 1H), 7.38 (ddd, J=8.6, 7.3, 1.7 Hz, 1H), 7.22-7.10 (m, 3H), 4.14 (t, J=6.4 Hz, 2H), 1.92 (h, J=7.0 Hz, 2H), 1.10 (t, J=7.4 Hz, 3H). ¹³C NMR (126 MHz, DMSO) δ 167.67, 155.55, 152.87, 151.45, 151.13, 144.75, 136.79, 130.71, 130.20, 126.35, 125.34, 124.93, 123.92, 123.30, 121.25, 118.40, 115.49, 113.35, 112.98, 70.16, 22.56, 11.27. HRMS (ESI) calcd for C₂₃H₁₉N₂O₆ [M+H]⁺ 419.1165; found 419.1237.

### 2-(5-(3-((tert-butoxycarbonyl)amino)phenyl)furan-2-yl)-6-nitroquinoline-4-carboxyli c acid (44)

¹H NMR (400 MHz, DMSO) δ 9.65 (d, J=2.7 Hz, 1H), 9.60 (s, 1H), 8.54 (s, 1H), 8.51 (dd, J=9.2, 2.6 Hz, 1H), 8.24 (d, J=9.2 Hz, 1H), 7.83 (d, J=8.6 Hz, 2H), 7.73 (d, J=3.8 Hz, 1H), 7.62 (d, J=8.5 Hz, 2H), 7.17 (d, J=3.7 Hz, 1H), 1.52 (s, 9H). ¹³C NMR (126 MHz, DMSO) δ 167.17, 157.05, 153.11, 152.82, 151.31, 145.80, 140.59, 138.78, 131.46, 125.65, 123.83, 123.53, 122.93, 122.63, 121.12, 118.93, 117.12, 108.89, 80.20, 28.72. HRMS (ESI) calcd for C₂₅H₂₂N₃O₇ [M+H]⁺ 476.1380; found 476.1452.

### 2-(5-(4-((tert-butoxycarbonyl)amino)phenyl)furan-2-yl)-6-nitroquinoline-4-carboxylic acid (45)

¹H NMR (400 MHz, DMSO) δ 9.68 (d, J=2.6 Hz, 1H), 9.56 (s, 1H), 8.54 (s, 1H), 8.52 (dd, J=9.2, 2.4, 1H), 8.25 (d, J=9.3 Hz, 1H), 8.02 (t, J=2.0 Hz, 1H), 7.74 (d, J=3.7 Hz, 1H), 7.52 (t, J=7.2 Hz 2H), 7.40 (t, J=7.9 Hz, 1H), 7.20 (d, J=3.7 Hz, 1H), 1.53 (s, 9H). ¹³C NMR (126 MHz, DMSO) δ 166.97, 156.75, 153.30, 151.79, 151.19, 145.69, 140.75, 139.07, 131.36, 130.12, 124.21, 123.26, 122.91, 120.67, 118.88, 116.71, 114.07, 109.87, 79.80, 31.43, 28.72. HRMS (ESI) calcd for C₂₅H₂₂N₃O₇ [M+H]⁺ 476.1380; found 476.1452.

### 2-(5-(benzo[d][1,3]dioxol-5-yl)furan-2-yl)-6-nitroquinoline-4-carboxylic acid (48)

¹H NMR (400 MHz, DMSO-d₆) δ 9.81 (d, J=2.7 Hz, 1H), 8.40 (dd, J=9.3, 2.7 Hz, 1H), 8.28 (s, 1H), 8.12 (d, J=9.3 Hz, 1H), 7.58 (d, J=3.6 Hz, 1H), 7.52-7.42 (m, 2H), 7.16 (d, J=3.6 Hz, 1H), 7.07 (d, J=8.1 Hz, 1H), 6.12 (s, 2H). ¹³C NMR (126 MHz, DMSO) δ 167.27, 156.02, 152.05, 151.17, 148.50, 148.22, 144.65, 130.67, 125.04, 124.44, 123.19, 118.89, 118.02, 115.54, 109.44, 108.61, 105.13, 101.94, 91.01, 68.65. HRMS (ESI) calcd for C₂₁H₁₃N₂O₇ [M+H]⁺ 405.0645; found 405.0716.

### 2-(5-(naphthalen-1-yl)furan-2-yl)-6-nitroquinoline-4-carboxylic acid (49)

¹H NMR (400 MHz, DMSO) δ 9.69 (d, J=2.6 Hz, 1H), 8.56 (s, 1H), 8.55-8.46 (m, 2H), 8.26 (d, J=9.3 Hz, 1H), 8.07 (d, J=8.0 Hz, 2H), 8.00 (dd, J=7.2, 1.2 Hz, 1H), 7.84 (d, J=3.6 Hz, 1H), 7.74-7.60 (m, 3H), 7.29 (d, J=3.6 Hz, 1H). ¹³C NMR (126 MHz, DMSO) δ 166.86, 156.45, 152.22, 151.31, 145.50, 139.39, 134.18, 131.46, 130.86, 130.18, 129.65, 129.31, 127.92, 126.85, 126.55, 125.65, 124.44, 123.83, 123.23, 120.21, 116.51, 113.49. HRMS (ESI) calcd for C₂₄H₁₅N₂O₅ [M+H]⁺ 411.0903; found 411.0975.

### 2-(5-([1,1′-biphenyl]-4-yl)furan-2-yl)-6-nitroquinoline-4-carboxylic acid (50)

¹H NMR (400 MHz, DMSO-d₆) δ 9.77 (d, J=2.7 Hz, 1H), 8.44 (dd, J=9.2, 2.8, 1H), 8.43 (s, 1H), 8.19 (d, J=9.2 Hz, 1H), 8.01 (d, J=8.8 Hz, 2H), 7.83 (d, J=8.8 Hz, 2H), 7.75 (dd, J=8.4, 1.6, 2H), 7.68 (d, J=3.6 Hz, 1H), 7.50 (t, J=7.2, 2H), 7.41 (t, J=7.2, 1H), 7.34 (d, J=3.7 Hz, 1H). ¹³C NMR (126 MHz, DMSO) δ 167.25, 156.02, 152.39, 151.17, 145.12, 140.50, 139.77, 131.01, 129.51, 127.75, 127.05, 125.26, 124.33, 123.60, 119.21, 117.73, 116.04, 109.98. HRMS (ESI) calcd for C₂₆H₁₇N₂O₅ [M+H]⁺ 437.1059; found 437.1131.

### 2-(5-([1,1′-biphenyl]-3-yl)furan-2-yl)-6-nitroquinoline-4-carboxylic acid (51)

¹H NMR (400 MHz, DMSO-d₆) δ 9.68 (d, J=2.6 Hz, 1H), 8.58 (s, 1H), 8.51 (dd, J=9.3, 2.7 Hz, 1H), 8.27 (d, J=9.4 Hz, 1H), 8.18 (t, J=2.0 Hz, 1H), 7.95 (dt, J=7.6, 1.4 Hz, 1H), 7.82-7.76 (m, 3H), 7.71 (dt, J=7.8, 1.4 Hz, 1H), 7.64 (t, J=7.8 Hz, 1H), 7.54 (t, J=7.2 Hz, 2H), 7.49-7.41 (m, 2H). ¹³C NMR (126 MHz, DMSO) δ 167.02, 164.96, 156.57, 152.04, 151.31, 145.64, 143.73, 141.80, 139.99, 132.37, 130.47, 129.49, 127.44, 123.36, 123.02, 122.93, 120.58, 120.51, 116.69, 111.00, 110.39. HRMS (ESI) calcd for C₂₆H₁₇N₂O₅ [M+H]⁺ 437.1059; found 437.1131.

### 6-nitro-2-(5-(4-phenoxyphenyl)furan-2-yl)quinoline-4-carboxylic acid (52)

¹H NMR (400 MHz, DMSO) δ 9.73 (d, J=2.6 Hz, 1H), 8.46 (dd, J=9.2, 2.6 Hz, 1H), 8.44 (s, 1H), 8.19 (d, J=9.2 Hz, 1H), 7.95 (d, J=8.7 Hz, 2H), 7.69 (d, J=3.6 Hz, 1H), 7.45 (dd, J=8.5, 7.3 Hz, 2H), 7.24-7.18 (m, 2H), 7.17-7.09 (m, 4H). ¹³C NMR (126 MHz, DMSO) δ 198.27, 167.30, 161.96, 157.68, 156.47, 151.78, 151.28, 145.26, 143.01, 130.70, 126.74, 125.10, 124.50, 123.85, 123.23, 119.55, 116.41, 109.08, 40.34, 37.55. HRMS (ESI) calcd for C₂₆H₁₇N₂O₆ [M+H]⁺ 453.1008; found 453.1081.

### 6-nitro-2-(5-(3-phenoxyphenyl)furan-2-yl)quinoline-4-carboxylic acid (53)

¹H NMR (400 MHz, DMSO) δ 9.87 (d, J=2.7 Hz, 1H), 8.39 (dd, J=9.2, 2.7 Hz, 1H), 8.24 (s, 1H), 8.11 (d, J=9.2 Hz, 1H), 7.72 (d, J=7.8 Hz, 1H), 7.60 (t, J=2.6 Hz, 2H), 7.54 (t, J=8.0 Hz, 1H), 7.48-7.39 (m, 2H), 7.32 (d, J=3.7 Hz, 1H), 7.19 (t, J=7.4 Hz, 1H), 7.14-7.05 (m, 2H), 7.00 (dd, J=8.2, 2.5 Hz, 1H). ¹³C NMR (126 MHz, DMSO) δ 167.47, 157.96, 157.05, 154.94, 153.43, 152.71, 151.29, 150.94, 146.41, 145.20, 143.20, 131.76, 130.86, 124.44, 124.21, 123.53, 123.30, 118.93, 117.79, 115.29, 114.70, 110.40, 39.96. HRMS (ESI) calcd for C₂₆H₁₇N₂O₆ [M+H]⁺ 453.1008; found 453.1082.

### 6-nitro-2-(5-(2-phenoxyphenyl)furan-2-yl)quinoline-4-carboxylic acid (54)

¹H NMR (400 MHz, DMSO) δ 9.85 (d, J=2.7 Hz, 1H), 8.40 (dd, J=9.3, 2.7 Hz, 1H), 8.31 (s, 1H), 8.19-8.11 (m, 2H), 7.57 (d, J=3.6 Hz, 1H), 7.47-7.36 (m, 4H), 7.16 (t, J=7.4 Hz, 1H), 7.11-7.01 (m, 4H). ¹³C NMR (126 MHz, DMSO) δ 167.22, 156.75, 152.71, 152.19, 151.69, 151.32, 150.94, 144.79, 130.55, 130.35, 127.46, 127.16, 125.12, 123.99, 123.08, 121.72, 119.98, 118.32, 118.16, 114.93, 113.69, 87.67, 54.99. HRMS (ESI) calcd for C₂₆H₁₇N₂O₆ [M+H]⁺ 453.1008; found 453.1081.

### General Procedure for the Production of Ethyl 2-(5-(4-butylphenyl)furan-2-yl)quinoline-4-carboxylates (2, 29)

A 2-(5-(4-butylphenyl)furan-2-yl)quinoline-4-carboxylic acid (50 μmol) was dissolved in ethanol (2 mL), stirred on ice, and H₂SO₄ (120 μL) was added dropwise. The reaction was warmed to 75° C. and stirred overnight (Scheme 2). The reaction was cooled to room temperature, and a sodium carbonate solution was added until the mixture was basic. The mixture was extracted with ethyl acetate, dried (MgSO₄), filtered, and concentrated in vacuo to afford the product.

### ethyl 6-bromo-2-(5-(4-butylphenyl)furan-2-yl)quinoline-4-carboxylate (2)

¹H NMR (400 MHz, DMSO-d₆) δ 8.77 (d, J=2.2 Hz, 1H), 8.42 (s, 1H), 8.06-7.95 (m, 2H), 7.82 (dd, J=4.7, 8.2 Hz, 2H), 7.60 (d, J=3.6 Hz, 1H), 7.34 (dd, J=3.7, 8.2 Hz, 2H), 7.20 (d, J=3.6 Hz, 1H), 4.53 (q, J=7.1 Hz, 2H), 2.65 (t, J=7.7 Hz, 2H), 1.60 (p, J=7.0, 8.5, 9.9 Hz, 2H), 1.46 (t, J=7.1 Hz, 3H), 1.35 (h, 2H), 0.93 (t, J=7.3 Hz, 3H). ¹³C NMR (126 MHz, DMSO-d₆) δ 188.57, 184.37, 169.27, 168.65, 168.16, 167.92, 151.73, 135.76, 134.16, 132.66, 132.46, 132.30, 132.19, 131.92, 129.51, 127.55, 124.65, 121.40, 119.58, 109.79, 62.55, 37.27, 34.98, 33.25, 22.08, 14.09. HRMS (ESI) calcd for C₂₆H₂₅BrNO₃ [M+H]⁺, 478.0940; found 478.10111.

### ethyl 2-(5-(4-butylphenyl)furan-2-yl)-6-nitroquinoline-4-carboxylate (29)

¹H NMR (400 MHz, Chloroform-d) δ 9.74 (d, J=2.5 Hz, 1H), 8.57 (s, 1H), 8.53 (dd, J=2.5, 9.2 Hz, 1H), 8.29 (d, J=9.5 Hz, 1H), 7.79 (d, J=8.0 Hz, 2H), 7.63 (s, 1H), 7.32 (d, J=8.0 Hz, 2H), 6.91 (d, J=3.6 Hz, 1H), 4.66 (q, J=7.1 Hz, 2H), 2.70 (t, J=7.7 Hz, 2H), 1.68 (p, J=7.6 Hz, 2H), 1.60 (t, J=7.1 Hz, 3H), 1.48-1.35 (m, 2H), 0.99 (t, J=7.3 Hz, 3H). ¹³C NMR (126 MHz, Chloroform-d) δ 172.17, 164.60, 159.70, 155.08, 150.83, 148.46, 147.46, 146.05, 144.28, 138.79, 137.91, 129.42, 127.13, 125.83, 122.96, 122.47, 119.95, 112.37, 104.91, 98.48, 88.49, 58.49, 35.57, 33.21, 22.19, 13.77. HRMS (ESI) calcd for C₂₆H₂₅N₂O₅ [M+H]⁺, 445.1685; found 445.17596.

### General Procedure for the Production of 2-(5-(aminophenyl)furan-2-yl)-6-nitroquinoline-4-carboxylic acids (46, 47)

A 2-(5-(4-((tert-butoxycarbonyl)amino)phenyl)furan-2-yl)-6-nitroquinoline-4-carboxylic acid (50 μmol) was dissolved in a 1 mL solution of 50:50 TFA:dichloromethane. The reaction stirred at room temperature and was monitored by TLC (95:5 dichloromethane:methanol). When the reactant was consumed, toluene (2 mL) was added, and the reaction was filtered and washed with methanol and diethyl ether to afford the product.

### 2-(5-(4-aminophenyl)furan-2-yl)-6-nitroquinoline-4-carboxylic acid (46)

¹H NMR (400 MHz, DMSO) δ 9.64 (d, J=2.6 Hz, 1H), 8.51 (s, 1H), 8.50 (dd, J=9.3, 2.7 Hz 1H), 8.21 (d, J=9.2 Hz, 1H), 7.70 (d, J=3.7 Hz, 1H), 7.63 (d, J=8.5 Hz, 2H), 6.96 (d, J=3.7 Hz, 1H), 6.71 (d, J=8.5 Hz, 2H). ¹³C NMR (126 MHz, DMSO) δ 181.43, 170.79, 167.47, 160.15, 151.45, 144.90, 138.48, 134.48, 130.86, 126.55, 125.65, 122.63, 116.21, 106.77, 99.75, 98.24, 96.36. HRMS (ESI) calcd for C₂₀H₁₄N₃O₅ [M+H]⁺ 376.0855; found 376.0927.

### 2-(5-(3-aminophenyl)furan-2-yl)-6-nitroquinoline-4-carboxylic acid (47)

¹H NMR (500 MHz, CDCl₃) δ 9.69 (d, J=2.6 Hz, 1H), 8.56 (s, 1H), 8.54 (dd, J=9.3, 2.7 Hz, 1H), 8.27 (d, J=9.2 Hz, 1H), 7.75 (dd, J=3.7, 0.9 Hz, 1H), 7.30-7.21 (m, 3H), 7.18 (dd, J=3.6, 1.2 Hz, 1H), 6.76 (d, J=6.8 Hz, 1H) 3.86 (s, 2H). ¹³C NMR (126 MHz, DMSO) δ 185.65, 184.23, 182.42, 166.86, 151.56, 145.80, 139.08, 131.36, 130.30, 124.44, 123.53, 123.22, 122.93, 121.12, 116.51, 109.79, 105.87, 103.07, 102.77. HRMS (ESI) calcd for C₂₀H₁₄N₃O₅ [M+H]⁺ 376.0855; found 376.0927.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An eIF4A inhibitor having a formula according to Formula I: 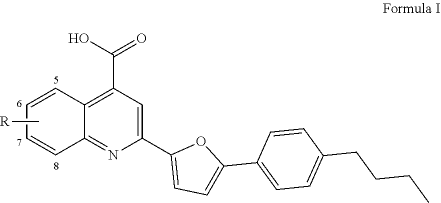
wherein R is H, alkyl, substituted alkyl, aryl, substituted aryl, heteroaryl, substituted heteroaryl, amino, alkoxy, nitro, or halo,
wherein the eIF4A inhibitor occupies a binding pocket of an eIF4A RNA-binding groove, thereby perturbing RNA binding to eIF4A, blocking ATP hydrolysis, and inhibiting eIF4A from unwinding RNA.

2. The eIF4A inhibitor of claim 1 having a formula according to Formula II:

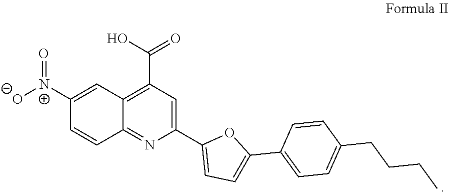 [SMILES: CCCCc1ccc(-c2ccc(-c3cc(C(=O)O)c4cc([N+](=O)[O-])ccc4n3)o2)cc1]

3. An eIF4A inhibitor having a formula according to Formula III: 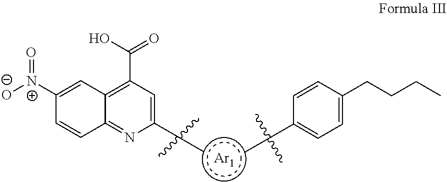
wherein Ar₁ is aryl, substituted aryl, heteroaryl, or substituted heteroaryl,
wherein the eIF4A inhibitor occupies a binding pocket of an eIF4A RNA-binding groove, thereby perturbing RNA binding to eIF4A, blocking ATP hydrolysis, and inhibiting eIF4A from unwinding RNA.

4. An eIF4A inhibitor having a formula according to Formula IV: 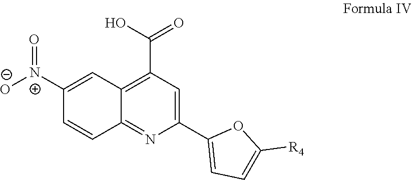
wherein R₄ is aryl, substituted aryl, heteroaryl, or substituted aryl,
wherein the eIF4A inhibitor occupies a binding pocket of an eIF4A RNA-binding groove, thereby perturbing RNA binding to eIF4A, blocking ATP hydrolysis, and inhibiting eIF4A from unwinding RNA.

5. The eIF4A inhibitor of claim 4 having a formula according to Formula V: 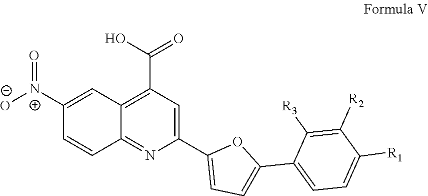
wherein R₁, R₂, and R₃ are each independently H, alkyl, substituted alkyl, alkoxy, aryl, or amino.

6. The eIF4A inhibitor of claim 4 having a formula according to Formula VI: 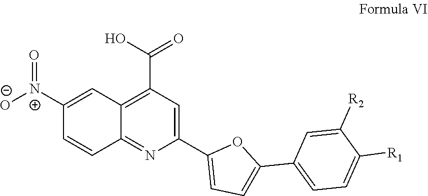
wherein R₁, and R₂ are each independently H, alkyl, substituted alkyl, alkoxy, aryl, or amino.

7. A method for inhibiting eIF4A activity in a cell, the method comprising:
a. contacting the cell with an eIF4A inhibitor having a formula according to Formula I: 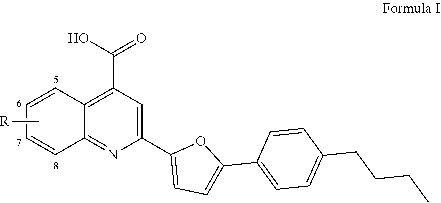
wherein R is H, alkyl, substituted alkyl, aryl, substituted aryl, heteroaryl, substituted heteroaryl, amino, alkoxy, nitro, or halo,
wherein the eIF4A inhibitor occupies a binding pocket of an eIF4A RNA-binding groove, thereby perturbing RNA binding to eIF4A, blocking ATP hydrolysis, and inhibiting eIF4A from unwinding RNA.

8. The method of claim 7, wherein the eIF4A inhibitor has a formula according to Formula II:

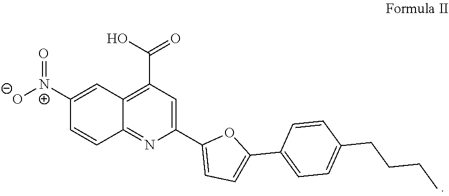 [SMILES: CCCCc1ccc(-c2ccc(-c3cc(C(=O)O)c4cc([N+](=O)[O-])ccc4n3)o2)cc1]

9. A method for inhibiting eIF4A activity in a cell, the method comprising:
a. contacting the cell with an eIF4A inhibitor having a formula according to Formula III: 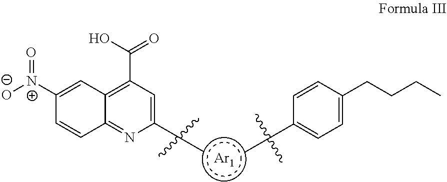
wherein Ar₁ is aryl, substituted aryl, heteroaryl, or substituted heteroaryl,
wherein the eIF4A inhibitor occupies a binding pocket of an eIF4A RNA-binding groove, thereby perturbing RNA binding to eIF4A, blocking ATP hydrolysis, and inhibiting eIF4A from unwinding RNA.

10. A method for inhibiting eIF4A activity in a cell, the method comprising:
a. contacting the cell with an eIF4A inhibitor having a formula according to Formula III: 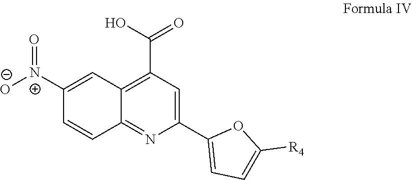
wherein R₄ is aryl, substituted aryl, heteroaryl, or substituted aryl,
wherein the eIF4A inhibitor occupies a binding pocket of an eIF4A RNA-binding groove, thereby perturbing RNA binding to eIF4A, blocking ATP hydrolysis, and inhibiting eIF4A from unwinding RNA.

11. The method of claim 10, wherein the eIF4A inhibitor has a formula according to Formula V: 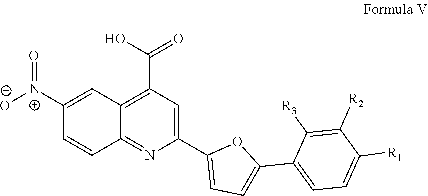
wherein R₁, R₂, and R₃ are each independently H, alkyl, substituted alkyl, alkoxy, aryl, or amino.

12. The method of claim 10, wherein the eIF4A inhibitor has a formula according to Formula VI: 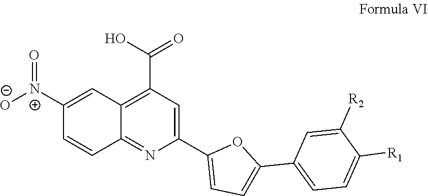
wherein R₁, and R₂ are each independently H, alkyl, substituted alkyl, alkoxy, aryl, or amino.
